SEQUENCE LISTING (15 sequences)

SEQ ID NO: 1 - DNA, 36 bp, Artificial Sequence
  Description of Artificial Sequence  Synthetic Primer
cctgaagacccagagatggccaagttgaccagtgcc

SEQ ID NO: 2 - DNA, 37 bp, Artificial Sequence
  Description of Artificial Sequence  Synthetic Primer
cctctctgggtcttcgtcctgctcctcggccacgaag

SEQ ID NO: 3 - DNA, 21 bp, Artificial Sequence
  Description of Artificial Sequence  Synthetic Primer
ccaccgccctcaaagtagacg

SEQ ID NO: 4 - DNA, 22 bp, Artificial Sequence
  Description of Artificial Sequence  Synthetic Primer
ccaacttaatcgccttgcagca

SEQ ID NO: 5 - DNA, 22 bp, Artificial Sequence
  Description of Artificial Sequence  Synthetic Primer
cgctacattactgttgcactcc

SEQ ID NO: 6 - DNA, 30 bp, Artificial Sequence
  Description of Artificial Sequence  Synthetic Primer
ggcctctgggtcttccgggtctccttctgc

SEQ ID NO: 7 - protein, 166 aa, Artificial Sequence
  Description of Artificial Sequence  Synthetic Peptide
WTVWFVLFVISVWDLVAVLTPKGPLRYLVETAQERNEPIFPALIYSSGVIYPYVLVTAVENTTDPREPTSSDSNTSTAFPGEASCSSETPKRPKVKRIPQKVQIESNTTASTTQNSGVRVERELAAERPTVQDANFHRHEEEERGVKLGLGDFIFYSVLLGKASSY

SEQ ID NO: 8 - protein, 124 aa, Artificial Sequence
  Description of Artificial Sequence  Synthetic Peptide
WTVWMALTAISFWDIVAVLTPCGPLKMLVETANRRGDDKFPAILYNSSSYVNEVDSPDTTRSNSTPLTEFNNSSSSRLLESDSLLRPPVIPRQIREVREVEGTIRLGMGDFVFYSLMLGNTVQT

SEQ ID NO: 9 - protein, 74 aa, Artificial Sequence
  Description of Artificial Sequence  Synthetic Peptide
WTWLILAISVYDLVAVLPKGPLRMLVETAQERNELFPALIYSSMVWVMSEEEERGVKLGLGDFIFYSVLVGKAT

SEQ ID NO: 10 - protein, 131 aa, Artificial Sequence
  Description of Artificial Sequence  Synthetic Peptide
WSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAAT

SEQ ID NO: 11 - protein, 129 aa, Artificial Sequence
  Description of Artificial Sequence  Synthetic Peptide
WSAWVILGAISVYDLLAVLCPKGPLRMLVETAQERNEPIFPALIYSSAMMWTVGMADSATADGRMNQQVQHIDRNTPEGANSTVEDAAETRIQTQSNLSSEDPDEERGVKLGLGDFIFYSVLVGKAAAT

SEQ ID NO: 12 - protein, 210 aa, Artificial Sequence
  Description of Artificial Sequence  Synthetic Peptide
WTAWAVLAAISIWDLIAVLSPRGPLRILVETAQERNEQIFPALIYSSTVVYALVNTVTPQQSQATASSSPSSSNSTTTTRATQNSLASPEAAAASGQRTGNSHPRQNQRDDGSVLATEGMPLVTFKSNLRGNAEAAGFTQEWSANLSERVARRQIEVQSTQSGNAQRSNEYRTVTAPDQNHPDGQEERGIKLGLGDFIFYSVLVGKASSY

SEQ ID NO: 13 - protein, 166 aa, Artificial Sequence; identical to SEQ ID NO: 7
  Description of Artificial Sequence  Synthetic Peptide

SEQ ID NO: 14 - protein, 124 aa, Artificial Sequence; identical to SEQ ID NO: 8
  Description of Artificial Sequence  Synthetic Peptide

SEQ ID NO: 15 - protein, 74 aa, Artificial Sequence; identical to SEQ ID NO: 9
  Description of Artificial Sequence  Synthetic Peptide

(19) United States
(12) Patent Application Publication
(10) Pub. No.: US 2003/0022151 A1
(43) Pub. Date: Jan. 30, 2003

(54) Functional screening

(76) Inventor: Gopal Thinakaran, Chicago, IL (US)

Correspondence Address: Priya D. Subramony, Fulbright & Jaworski L.L.P., Suite 2400, 600 Congress Avenue, AUSTIN, TX, 78701, US

(21) Appl. No.: 10/051,767

(22) Filed: Jan. 17, 2002

Related U.S. Application Data
(60) Provisional application No. 60/262,353, filed on Jan. 17, 2001.

(51) Int. Cl. C12Q 1/00; C12Q 1/68; G01N 33/53; G01N 33/567

(52) U.S. Cl. 435/4; 435/6; 435/7.2

(57) ABSTRACT

Screening methods for identifying molecules that interact with and/or regulate accumulation and stabilization of unstable proteins are provided. The screening methods involve generation of chimeric proteins comprising a region of an unstable protein linked to a region of a marker gene product. Changes in the levels of the unstable protein, due to accumulation, and/or stabilization and/or hyperaccumulation are then determined by analyzing the change in the marker gene product level. The marker gene product can be an antibiotic resistance gene that confers antibiotic resistance by stoichiometrically binding to the antibiotic or it can be a fluorescent protein region. Thus, antibiotic resistance screening or fluorescence imaging or cell sorting methods can be used to detect changes in the levels of the unstable chimeric proteins. Unstable proteins suitable for this screening method include, but are not limited to, membrane proteins such as ion channels, receptors, or presenilins.

The present application is a continuation-in-part of co-pending U.S. patent application Ser. No. 60/262,353, filed Jan. 17, 2001. The entire text of the above-referenced disclosure is specifically incorporated by reference herein without disclaimer.

### BACKGROUND OF THE INVENTION 1. Field of the Invention

The present invention relates generally to screening methods for identifying molecules that interact with and regulate levels of proteins of interest. More particularly, it concerns the development of screening methods to identify molecules that regulate the accumulation and/or stabilization of unstable proteins, for example, proteins such as presenilins, which are proteins involved in the pathogenesis of Alzheimer's Disease, as well as of several other membrane spanning proteins including those that are comprised of protein subunit associations.

2. Description of Related Art

Alzheimer's disease (AD) is the most common type of progressive dementia in the elderly. AD is characterized by initial memory loss, followed by progressive loss of neurons leading to dementia and loss of all nervous functions, and eventually death. AD is now the fourth-largest killer of adults 65 and older, and this disease impacts about one of every three families in the United States, and affects over 13 million people world-wide. As the population trends lead to an increase in the number of older people, this figure will only increase.

A subset of AD is classified as familial early-onset AD (FAD) where onset of the disease begins as early as the fourth to sixth decade of life. FAD is an inherited autosomal dominant disorder. Mutations in genes encoding polytopic membrane proteins called presenilins (PS), exemplified by PS1 and PS2, co-segregate with the majority of pedigrees with early-onset AD.

AD is pathologically characterized by the presence of neurofibrillary tangles and the cerebral deposition of β-amyloid (Aβ) peptides that are 40-42 amino acids in length and are derived from the amyloid precursor protein (APP). Two lines of evidence demonstrate the involvement of PS (PS1 and PS2) in Aβ production. First, Aβ production is abrogated in PS1 deficient (PS1^{-/-}) cells. Second, FAD-linked mutant PS1 increases the production of highly fibrillogenic Aβ42 peptides. The PS proteins mediate the cleavage of APP and also facilitate proteolytic processing of other transmembrane (TM) proteins such as APP homologues, APLP1, Notch 1, Irel, and likely other yet unidentified proteins. However, the precise role of PS1 in Aβ production, and the molecular mechanisms by which FAD-linked PS1 mutations lead to elevations in Aβ42 production are not yet defined. In addition, very little information is available regarding the molecular and structural domains of PS1 critical for these biological functions.

Mutations in genes encoding PS1 and PS2 are responsible for ˜50% of pedigrees with FAD. In the past five years, several laboratories have focused on investigating PS metabolism, PS biological functions, and the mechanisms by which mutant PS proteins promote AD pathogenesis. Analysis of PS1-null mice revealed an essential role for PS1 in embryonic development, and biochemical analysis of cells derived from PS1-embryos uncovered important role(s) for PS in facilitating the trafficking/cleavage of a set of membrane-bound proteins, including: the amyloid precursor protein (APP); amyloid precursor-like protein 1; the receptor tyrosine kinase TrkB; and Notch1. Mutant PS proteins selectively increase the levels of highly fibrillogenic Aβ42 species in cultured cells and in vivo, and accelerate Aβ deposition in the brains of transgenic mice. This gain-of-function of FAD-linked mutant PS in enhancing Aβ42 production appears, on face value, distinct from the role of PS1 in development. For example, expression of PS1 bearing FAD-linked mutations can rescue the embryonic lethality of PS1 null mutations. Recent studies have disclosed PS1 interactions with several proteins including members of the armadillo family of proteins (Yu et al., 1998; Zhou et al., 1997; Murayama et al., 1998; Stahl et al., 1999; Levesque et al., 1999; Tanahashi and Tabira, 1999).

Although other laboratories have identified several proteins that interact with the PS proteins using methods such as the yeast-two-hybrid method and candidate interaction techniques; the influence of these interacting proteins on PS metabolism, and/or on the accumulation of PS fragments, and/or on enhanced production of amyloid in FAD mutants has not been described. Therefore, there are additional as yet unidentified proteins that interact with PS proteins and mediate excessive accumulation of PS proteins, leading to the pathology of AD. Hence, there is an unmet need in the art to identify such proteins. Identification of proteins that regulate the accumulation of PS fragments will be of great benefit in understanding the molecular basis for AD. This understanding will lead to better and more effective treatments for AD which will be a boon to the ever increasing population afflicted with AD.

### SUMMARY OF THE INVENTION

The present invention overcomes these and other deficiencies in the art and provides screening methods for the identification of proteins and other molecules that cause the accumulation or stabilization of proteins of interest. Stabilization or accumulation may be mediated by interaction with the protein of interest. A “protein of interest” is defined herein as any protein that is or is generally associated with any other protein and/or polypeptide subunit and/or other molecule. The association may be required to provide functionality, to confer stability, to promote proper maturation and subcellular localization, or to prevent degradation of the protein. A “protein of interest” also includes proteins that interact with other proteins and/or proteins that interact with other subunits, such as homomeric or heteromeric subunits, of the same protein In some aspects, the protein of interest is an unstable protein. An “unstable protein” is defined herein as a protein that requires association(s) with other molecule(s) to prevent its intracellular degradation. Unstable proteins are degraded by cellular mechanisms in the absence of association(s) with other proteins and thus, on their own these proteins have a very short half life. In some examples, association with other molecules enables the unstable protein to be targeted to different cellular destinations, such as to the endoplasmic reticulum for packaging/transport etc. In yet other cases, associations with other molecules, which can be other proteins, leads to the formation of a protein complex comprising several protein subunits and this is required for protein function. One example of this type of a protein formed by subunit associations is the nicotinic acetylcholine receptor (N-AChR). Other examples of unstable proteins include membrane proteins such as ion channels and receptor proteins. For example, polytopic membrane proteins with complex structure comprising co-factors such as, ligand-gated ion channels which are exemplified by the nicotinic acetylcholine receptors, the GABA receptors, the glycine receptors, etc.; the voltage-gated ion channels which are exemplified by the voltage-gated Na⁺ channels, the voltage-gated K⁺ channels, and the voltage-gated Ca²⁺ channels; and other membrane proteins such as the presenilin (PS) are all examples of unstable proteins.

Thus, the invention provides methods for identifying candidate substances that change the levels of accumulation of a protein comprising a) obtaining a cell expressing a chimeric polypeptide comprising a polypeptide region of the protein linked to at least one marker gene product region; b) exposing the cell to a candidate substance; and c) determining any change in a level of the chimeric protein subsequent to exposing the cell to the candidate substance. In some embodiments, the methods further comprise assaying the level of the chimeric protein using the marker gene product.

In some embodiments, the protein is an unstable protein. In particular embodiments, the unstable protein is a presenilin protein, an amyloid precursor protein, or an amyloid precursor protein derivative. Specific examples of presenilin proteins include PS1 and/or PS2.

In other embodiments, the unstable protein is a polytopic membrane protein. Polytopic membrame proteins are defined as proteins that traverse or pass the plasma membrane more than one times. For example presenilins pass the membrane eight times. Other polytopic proteins or multipass proteins or membrane spanning proteins are well known to the skilled artisan. In some specific aspects, the polytopic protein is further defined as being comprised of a complex which comprises at least one co-factor. Examples of co-factors include, 1) the sodium-hydrogen exchanger regulatory factor (NHERF), which is a critical cofactor for rabbit kidney sodium-hydrogen exchanger, beta2-adrenergic receptor, the platelet-derived growth factor receptor, cystic fibrosis transmembrane conductance regulator and the sodium-bicarbonate transporter; 2) toll-like receptor 4 (TLR4), which is a membrane cofactor for the GPI-linked protein, CD 14.

In other specific aspects of the method, the unstable protein is a ligand-gated ion channel or a voltage-gated ion channel. In yet other specific aspects, the ligand-gated ion channel is exemplified by a nicotinic acetylcholine receptor, a GABA receptor, or a glycine receptor. In still other specific aspects, the unstable protein is a voltage-gated ion channel and is exemplified by a voltage-gated Na²⁺ channel, a voltage-gated K⁺ channel, or a voltage-gated Ca²⁺ channel.

In other embodiments of the methods, the change in the level of protein is either an increase or a decrease in the level of accumulation of the protein.

The methods of the invention allow the screening of a variety of candidate substances including chemical compounds, proteins, polypeptides, peptide mimetics, pharmacological compounds, nucleic acids, and the like. In some aspects, the candidate nucleic acid may be a cDNA or a genomic DNA that encodes a protein.

In aspects where candidate nucleic acids are screened, the method further comprises transfecting a cell that expresses a chimeric polypeptide that comprises a polypeptide region of the protein of interest linked to a marker gene region with the nucleic acid to expose the cell to the nucleic acid and hence the mRNA and/or protein encoded by it. This functional cloning method allows the screening of a variety of candidate nucleic acids. In further aspects of this method, the nucleic acid candidates that change the levels of the proteins of interest are identified and their corresponding proteins are identified. In yet other aspects, these identified proteins are further isolated and further characterized. Methods for identification, isolation and characterization of proteins that correspond to a known nucleic acid sequence are well known in the art and are also described elsewhere in this specification.

In some embodiments, the cell is contacted with a candidate substance. In some aspects, the contacting comprises injecting the cell with the candidate substance. In yet other embodiments, the contacting comprises administering the candidate substance to the cell.

In some embodiments, the marker gene product that is linked to the protein of interest is a fluorescent gene product. In specific embodiments, the fluorescent gene product is a green fluorescent protein, a yellow fluorescent protein, a blue fluorescent protein, or a red fluorescent protein. One of skill in the art will recognize that any fluorescent gene product may be used including a naturally occurring species, a mutational variant, a variant with enhanced fluorescent properties and the like. When the marker gene product is a fluorescent protein, changes in the expression of the chimeric protein comprising such a protein, are determined by fluorescence measurements, fluorescence imaging, or cell sorting. These methods are well known to the skilled artisan.

In other embodiments, the marker gene product is an antibiotic resistance gene product. In specific embodiments, the antibiotic resistance gene product is further defined as one that confers antibiotic resistance by binding stoichiometrically to an antibiotic. In other specific embodiments, the antibiotic resistance gene product is selected from the group comprising a bleomycin resistance gene product, a zeocin resistance gene product, a zorbamycine resistance gene product, a victomycin resistance gene product, a platomycin resistance gene product, a tallysomycin resistance gene product, a SF 1771 resistance gene product, a SF 1961 resistance gene product, or a YA 56 resistance gene product. In some embodiments, the antibiotic resistance gene product is the bleomycin resistance gene product. In yet other embodiments, the chimeric protein further comprises another marker gene such as a fluorescent protein gene that is attached to allow for visualization.

Therefore, in some embodiments, determining a change in the level of the chimeric protein comprises an antibiotic selection assay. Cells that accumulate increased levels of the chimeric proteins are the cells that have been exposed to a candidate substance that allows for or increases the accumulation of the unstable protein. Such cells are selected in a medium containing higher concentration of the antibiotic.

The invention also provides methods for identifying candidate substances that change the levels of accumulation of an unstable protein comprising a) obtaining a cell expressing a chimeric polypeptide comprising a polypeptide of the unstable protein linked to a marker gene product; b) exposing the cell to a candidate substance; and c) determining any change in the level of the chimeric protein subsequent to exposing the cell with the candidate substance. In some embodiments, the methods further comprise assaying the level of the chimeric protein using the marker gene product. In other embodiments, the protein is a presenilin protein and can be PS 1 or PS2.

In some embodiments, the marker gene product is an antibiotic resistance gene product. In some specific aspects, the antibiotic resistance gene product is a bleomycin resistance gene product. In other specific aspects, the bleomycin resistance gene product is the Ble protein.

In alternative embodiments, the marker gene product is a fluorescent gene product such as a green fluorescent protein, a yellow fluorescent protein, a red fluorescent protein, or a blue fluorescent protein. Therefore, determining changes in the levels of the chimeric protein comprises measuring the level of the fluorescent gene product by methods well known in the art.

The invention also provides methods for identifying candidate substances that change the levels of accumulation of a presenilin protein comprising a) obtaining a cell expressing a chimeric presenilin polypeptide comprising a presenilin polypeptide linked to a bleomycin resistance gene product; b) exposing the cell to a candidate substance; and c) determining any change in a level of the chimeric presenilin subsequent to exposing the cell with the candidate substance. In some aspects, the determining comprises an antibiotic selection assay. In specific aspects, the antibiotic is bleomycin. In other aspects, the antibiotic selection assay is performed at varying concentrations of bleomycin ranging from low concentrations, to select for low levels of chimeric protein, to higher concentrations of bleomycin, to select for cells that have higher levels of chimeric protein.

The invention also provides methods for identifying candidate substances that change the levels of accumulation of a presenilin protein comprising a) obtaining a cell expressing a chimeric presenilin polypeptide comprising a presenilin polypeptide linked to a fluorescent protein gene product; b) exposing the cell to a candidate substance; and c) determining any change in a level of the chimeric presenilin subsequent to exposing the cell with the candidate substance. In specific aspects of this method the determining comprises measuring changes in the fluorescence of the fluorescent protein. Although any fluorescent gene product may be used in certain specific embodiments the fluorescent gene product can be a green fluorescent protein, a yellow fluorescent protein, a red fluorescent protein, or a blue fluorescent protein.

The candidate substances identified by the screening methods set forth herein will be useful in developing agents that will prevent and/or provide therapeutic relief for pathologies that involve accumulation of proteins.

As used herein the specification, “a” or “an” may mean one or more. As used herein in the claim(s), when used in conjunction with the word “comprising”, the words “a” or “an” means one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Structure of PS1.

FIG. 2. The influence of mutant PS1 and PS2 on Aβ42 production. COS cells were co-transfected with cDNA encoding human APPswe and wild type PS1, PS1M146L, wild type PS2, or N141I. The levels of Aβx-40 and Aβx-42 in the conditioned medium were quantified by two-site ELISAs. Mean values±S.E. from two independent experiments are shown. *P=0.0012, **P<0.001, relative to values from cells transfected with the corresponding wild-type cDNA.

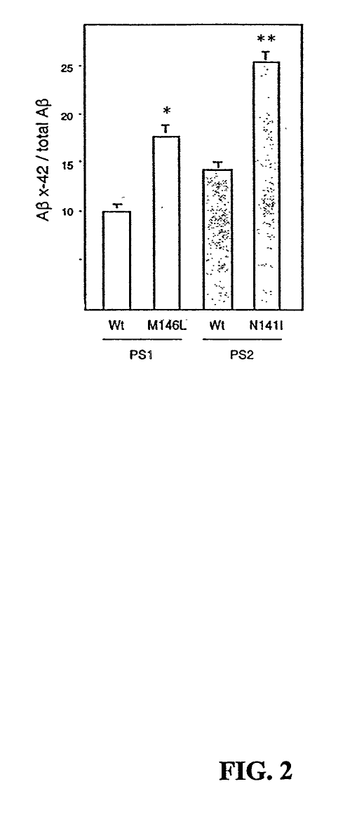

FIG. 3. Model for PS metabolism and saturable accumulation of derivatives.

FIG. 4. Schematic representation of PS1 deletion mutant PS1AHL, and the chimeric derivative, PS1ΔBle. Sh ble-encoded residues are shown as a stippled box.

FIG. 5. ClustalW alignment of the “loop” domain of PS1 and homologues. HuPS1 (SEQ ID NO:7), Xen PSα (SEQ ID NO:8), Fish PS1 (SEQ ID NO:9), Hu PS2 (SEQ ID NO:10), Xen PSβ (SEQ ID NO:11), Dros PS1 (SEQ ID NO:12), SEL-12 (SEQ ID NO:13), HOP-1 (SEQ ID NO:14), Consensus (SEQ ID NO:15).

FIG. 6. Influence of HL deletion on Aβ production in COS cells. COS cells were transfected with PS1, PS1ΔHL, PS2, or PS2ΔHL cDNA (Wt or FAD mutants as indicated) along with cDNA encoding APPswe and conditioned medium was collected 48 after transfection. The amount of secreted Aβx-40 and Aβx-42 was quantified from using two-site ELISAs and Aβx-42/total Aβ ratio (mean±S.E. of three transfections) was calculated. PS1 Wt versus PS1 mutants P<0.0001; PS1ΔHL versus PS1ΔHL mutants P=0.0026; PS2 Wt versus PS2 mutants P=0.0055; PS2ΔHL versus PS2ΔHL mutants, 16.57±1.16; P=0.0021.

FIG. 7. Quantification of Aβ production in double stable N2a cell lines. Quantitative analysis of secreted Aβ×-40 and Aβx-42 in the media conditioned by double stable cell lines was performed using two-site ELISAs. The ratio of Aβx-42/total Aβ was calculated; mean±S.E. from two independent experiments (7 samples) are shown. Deletion of the HL domain does not affect the elevated production of Aβ42 by mutant polypeptides. *, P<0.0001, relative to values from cells transfected with the corresponding Wt cDNA.

FIG. 8. A PS1-Sh ble chimera that is attached to the yellow flourescent protein (YFP) for visualization.

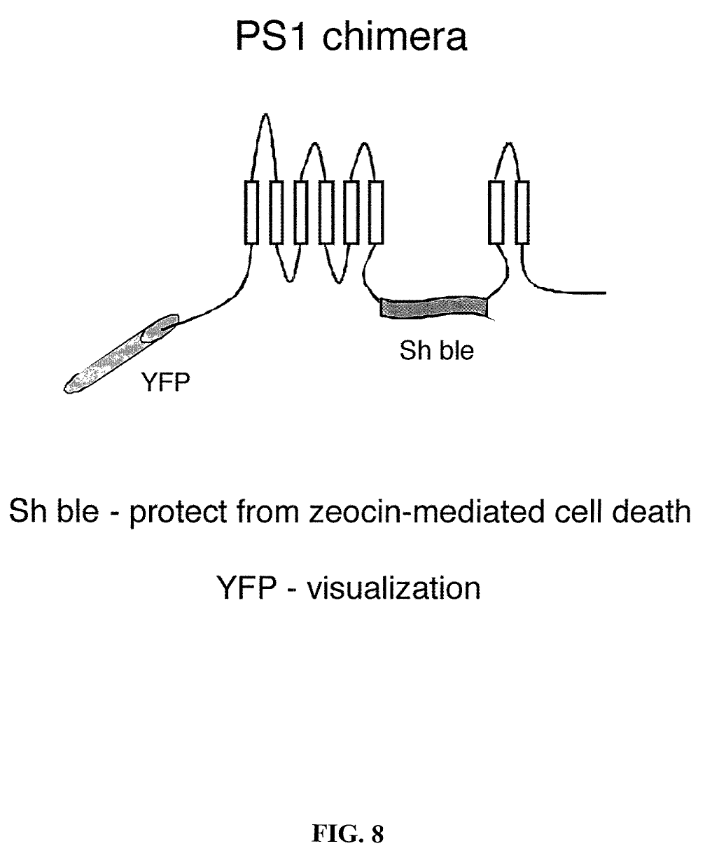

FIG. 9. Effect of deletion of transmembrane domains 2 and 3 on PS1 endoproteolysis, stability ands function.

FIG. 10. Stable chimeric PS1 polypeptides confer antibiotic resistance.

FIG. 11. Cell viability assay of culture stable N2a cells expressing PS1 chimeras in a multiwell format in medium containing different concentrations of zeocin. Inhibitors that affect the levels of PS1-Sh ble will reduce the viability of cells.

FIG. 12. Zeocin toxicity curve. Parental N2a cells and stable clones expressing a PS1 chimeric polypeptide were exposed to increasing concentrations of zeocin for three days and cell viability was measured using ELISA.

FIG. 13. Treatment with an inhibitor of presenilins which reduces the levels of the PS1-Sh ble polypeptide, such as L-685,458, reduced the resistance to zeocin.

FIG. 14. Functional cloning for screening candidate nucleic acids.

### DESCRIPTION OF ILLUSTRATIVE EMBODIMENTS

The Present Invention

Although methods based on yeast two-hybrid systems and candidate interaction techniques have been developed to determine protein-protein interactions these methods have proven ineffective in the identification of proteins that regulate the accumulation and/or deposition and/or stabilization of unstable proteins, for example, proteins such as the presenilins. As described above, unstable proteins are proteins that require association(s) with other molecules. In the absence of such association(s), these unstable proteins are degraded in the cell. Often association with other molecules enables the unstable protein to translocate within the cell and reach different cellular destinations, such as the endoplasmic reticulum for packaging/transport etc. In yet other cases, associations with other molecules, which can be other proteins, leads to the formation of a protein comprising several protein subunits and this is required for protein function. One example of this type of a protein formed by subunit associations is the nicotinic acetylcholine receptor (N-AChR).

The hyperaccumulation of some unstable proteins has been documented in several pathologies. For example, accumulation of presenilin proteins (PS) has been shown to increase the production of fibrillogenic amyloid deposits that lead to Alzheimer's disease. While yeast-two hybrid and candidate interaction methods have identified several proteins that can bind to the PS proteins none of these proteins have been shown to regulate/control the accumulation or stabilization of PS proteins.

The present invention provides novel screening methods that identify candidate substances that regulate or control or change the accumulation and/or stabilization of unstable proteins, protein subunits, and/or protein fragments. The screening methods of the present invention comprise a) obtaining a cell expressing a chimeric polypeptide comprising a polypeptide region of an unstable protein linked to a marker gene product region; b) exposing the cell to a candidate substance; and c) determining any change in the level of the chimeric protein subsequent to exposing the cell with the candidate substance. One may further assay or quantitate the marker gene product to determine the level of the chimeric protein.

Various candidate substances can be identified by this method. The candidate substances may be proteins, polypeptides, nucleic acids, chemical compounds, or pharmaceutical compounds.

The marker gene product can encode a fluorescent protein, for example a green fluorescent protein (GFP) sequence or a yellow fluorescent protein (YFP) sequence. Alternatively, the marker gene product can be an antibiotic resistance gene product. The antibiotic resistance gene product contemplated here is one that confers antibiotic resistance by binding stoichiometrically to an antibiotic in an antibiotic screening assay. Therefore, if a candidate substance increases the accumulation of the chimeric protein that comprises the antibiotic resistance gene product, a cell expressing this chimeric protein can now be selected in a medium containing higher antibiotic concentration than prior to interaction with the candidate substance. For example, the bleomycin resistance gene (ble) encodes a protein, the bleomycin resistance protein (Ble), which provides antibiotic resistance by binding stoichiometrically with bleomycin. Thus, by the methods of the present invention, a) transfection of a cell with the chimeric PS/Ble polypeptide; followed by b) contacting/exposing/administering a candidate substance that can change the level of PS accumulation; and c) selecting cells that have an increase in PS accumulation at a higher concentration of the antibiotic allows the identification of a candidate substance that increases the accumulation of PS proteins. This also allows identification of candidate substances that lead to enhanced stabilization and/or hyperaccumulation of PS proteins.

Thus, the “chimeric proteins” of the present invention are defined as chimeric proteins or fusion proteins that comprise a region of an unstable protein linked to a region of a marker gene product. Hence, “chimeric protein constructs” of the invention refer to nucleic acid molecules that encode the chimeric proteins.

The invention also describes the development of screening methods to screen candidate nucleic acids to identify genes and proteins that regulate the levels of accumulation of proteins of interest. For example, a cDNA library-based expression cloning system has been developed where a cDNA from a library is used co-transfect cells expressing the chimeric proteins of the invention. Thus, one can identify if the genes or protein that is encoded by the cDNA causes the accumulation or stabilization of unstable proteins. The nucleic acids or fragments thereof can then recovered using PCR based amplification of the vector cDNA insert. One can also identify and isolate the corresponding protein. Such methods provide high-throughput screening assays for screening a variety of nucleic acids to identify candidate molecules that can regulate levels and accumulation of proteins.

Stable mammalian brain cell lines have been generated that express and processes unstable proteins normally and therefore provide a cellular environment that mimics in vivo cellular environments where unstable proteins are normally processed. In the case of the PS proteins, the N2a neuroblastoma cell line is an example of a cell line that processes PS proteins as do normal cells and hence is a good model system for identifying proteins and nucleic acids that regulate PS accumulation and stabilization.

The inventors have also demonstrated that the chimeric proteins of the invention are functional in cells. For example, the PS protein chimeras were analyzed by functional assays that assayed the intramembraneous cleavage of amyloid precursor protein (APP) and Notch1. Both PS1 and PS2 are required for the APP and Notch1 cleavage. Therefore, APP and Notch processing were examined in PS1−/− fibroblasts that were transfected with expression plasmids encoding chimeric PS proteins along with a cDNA encoding APP or Notch1. Processing of APP was monitored by the secretion of Aβ. Notch processing was monitored by the generation of the Notch intracellular domain (NICD). As PS1−/− fibroblasts completely lack PS1, they do not produce either Aβ or NICD. Transfection with wild-type PS1 “rescues” this deficiency. The chimeric PS polypeptides of the present invention were also efficient in rescuing Aβ and NICD production in transfected PS1−/− fibroblasts. Thus, the chimeric PS polypeptides are functional in a cell culture-based assay which, as known to one of ordinary skill in the art, is widely used to study PS function.

Therefore, the present invention offers several advantages over yeast two-hybrid systems: 1) mammalian cell lines expressing a chimeric unstable polypeptide, which as shown for PS proteins, undergo regulated endoproteolysis and generate stable PS derivatives provide a cellular environment that allows identification of cellular proteins found in mammalian systems; and 2) the co-transfection method using a cDNA library-mediated expression cloning system provides a positive functional selection for proteins that participate in regulating the accumulation of unstable proteins such as stable PS protein derivatives.

Thus, proteins that regulate PS accumulation, by increasing stability of PS proteins, by providing inter or intra-molecular associations that enhance PS accumulation or by any other means can be identified by the methods disclosed herein. The present inventors envision that the identity of proteins and/or chemical compounds that regulate PS accumulation, given documented role of PS in increased Aβ production, will be valuable for the development of novel therapies for prevention and cure of AD. Therapies aimed at modulating the gain of function propert(ies) of FAD-linked mutant PS proteins will be benefited. Thus, the methods will provide valuable information for the design of rational therapeutic strategies to reduce Aβ burden. As will be recognized by one of skill in the art, the methods provided here are not limited to the PS proteins which are used as an example herein and in fact enable the identification of various nucleic acids, proteins and/or chemical compounds that regulate the stabilization and accumulation of other unstable proteins, polytopic proteins, as well as other proteins that are formed by the association of other proteinaceous subunits and/or other molecules.

A. Presenilins and Presenilin Metabolism

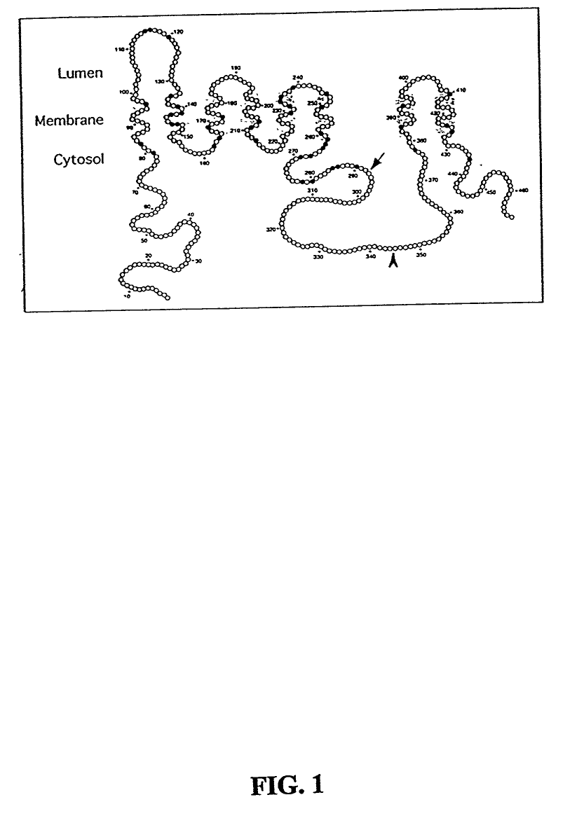

PS1 is a 467 amino acid polypeptide predicted to contain 8 transmembrane (TM) spanning domains with its N-terminus, C-terminus and a large hydrophilic “loop” region located between TMs 6 and 7 all oriented towards the cytosol (FIG. 1) (Doan et al, 1996; Li and Greenwald, 1996; Li and Greenwald, 1998; Lehmann et al., 1997). Although PS1 is synthesized as a 42- to 43-kD polypeptide, the preponderant PS1-related species that accumulate in vivo are 27- to 28-kD N-terminal (NTF) and 16- to 17-kD C-terminal (CTF) proteolytic derivatives (Thinakaran et al., 1996; Mercken et al, 1996). These PS1 derivatives are generated by endoproteolyis at Met 292 (and to a lesser extent, Met 298) within the cytoplasmic “loop” domain between TMs 6 and 7 (Thinakaran et al., 1996; Podlisny et al., 1997; Steiner et a/., 1999). Consistent with this finding, the FAD-linked PS1ΔE9 variant, which lacks 29 residues encoded by exon 9 (amino acids 291-319), fails to be cleaved (Thinakaran et al., 1996). However, lack of cleavage in PS1ΔE9 appears to be an exception, because FAD-linked missense PS1 variants do not affect endoproteolysis in cultured cells, transgenic mice, or in the brains of patients with FAD (Podlisny et al., 1997; Borchelt et al., 1996; Duff et al, 1996). At present, neither the identity of the protease nor the physiological significance of PS1 proteolysis is known. In addition to the endoproteolytic processing described above, PS1 and PS2 also undergo additional cleavage within the hydrophilic loop domain, dubbed “alternative cleavage”, that is mediated by caspases (Kim et al., 1997; Loetscher et a!., 1997; Grunberg et al., 1998).

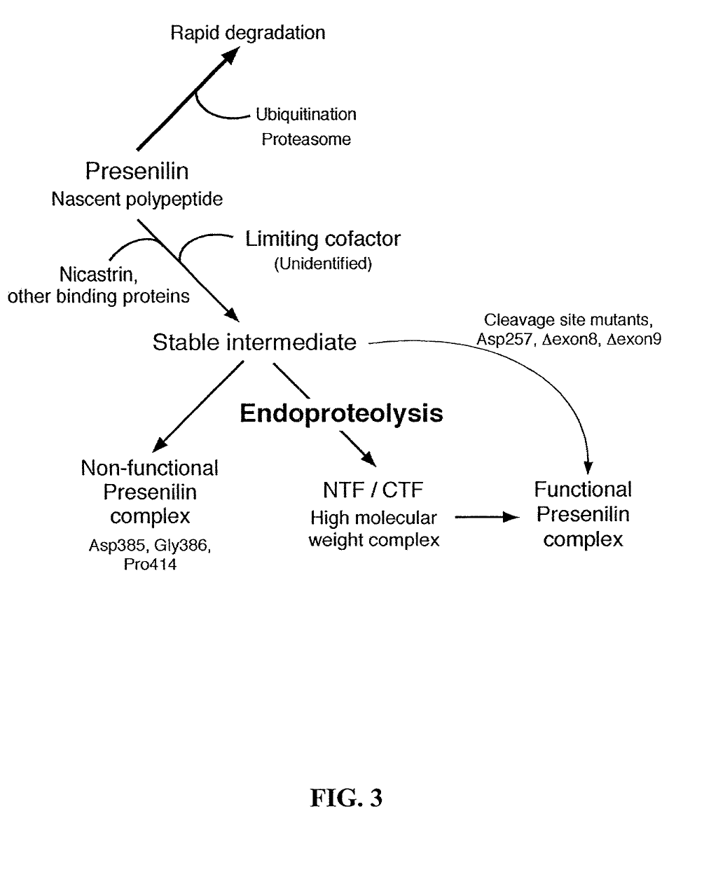

Analysis of human PS1 expression in brains of transgenic mice revealed a highly unusual aspect of the metabolism of presenilins (FIG. 3). The levels of PS1 derivatives are remarkably disproportionate to levels of transgene-derived mRNA or full-length human PS1 (Thinakaran et al., 1996). Similarly, in transfected cells only a small fraction of newly synthesized full-length PS1 and PS2 is converted to stable fragments, whereas the majority of the overexpressed protein fails to be processed and is rapidly degraded (Ratovitski et al, 1997; Thinakaran et al., 1997; Kim et al., 1997; Zhang et al., 1998). The saturable accumulation of PS1 NTF and CTF appears to be regulated by a post-translational mechanism (Thinakaran et al., 1997). Remarkably, in mouse N2a cell lines and in brains of transgenic mice expressing human PS1, accumulation of human PS1 derivatives is accompanied by a compensatory and highly selective decrease in the steady-state levels of murine PS1 and PS2 derivatives (Thinakaran et aL, 1996; Thinakaran et al., 1997). Similarly, the levels of murine PS1 derivatives are diminished in cultured cells overexpressing human PS2. Overexpression of the PS1ΔE9 variant, which fails to be cleaved, also resulted in compromised accumulation of murine PS1/PS2 derivatives suggesting that endoproteolysis is unlikely to be a limiting reaction that regulates “replacement” of murine PS1 and PS2 by overexpressed human PS1. These results are consistent with a model in which the abundance of PS1 and PS2 derivatives is coordinately regulated by competition for shared, but limiting, cellular factor(s) (Thinakaran et al., 1997). In view of the demonstration of a paucity of full-length PS1 and highly regulated accumulation of processed derivatives in vivo, the inventors and others (Ratovitski et al., 1997; Thinakaran et al., 1997; Kim et al., 1997; Zhang et al., 1998) have concluded that it is highly likely that PS1 fragments are the “functional units”.

Presenilins (PS), including PS1 and PS2, are involved in the pathogenesis of Alzheimer's Disease (AD) and mutations in presenilins have been associated in 50% of pedigrees with familial early-onset AD (FAD). For example, PS1 is necessary for γ-secretase cleavage of the Alzheimer's precursor protein (APP) and during APP cleavage FAD-linked PS 1 and PS2 mutants have been shown to selectively enhance the production of the Aβ42 peptides in transfected mammalian cells, in the brains of transgenic mice, as well as in patients with AD (Borchelt et al., 1996; Duff et al., 1996; Scheuner et al., 1996; Tomita et al., 1997; Borchelt et al., 1997; Holcomb et al., 1998). The Aβ42 peptides are more fibrillogenic than the shorter Aβ40 peptides and thus, FAD-linked mutant PS proteins lead to the production of highly amyloidogenic Aβ species which leads to Aβ deposition in brain.

As described above, PS1 undergoes endoproteolytic processing in vivo to generate stable (t_{½}˜24 h) NTF and CTF. The present inventors have shown by co-immunoprecipitation and chemical cross-linking studies that these two fragments remain associated (Suzuki et al., 1994). The inventors have also shown that a polypeptide corresponding to human NTF does not associate with endogenous mouse CTF (Tomita et al., 1999) and that fragments derived from endoproteolysis of co-expressed PS1 and PS2 fail to form heteromeric PS1·PS2 complexes. Nevertheless, fragments derived from a chimeric polypeptide corresponding to PS1NTF/PS2CTF form PS1NTF/PS2CTF assembly. Thus, full-length PS polypeptides establish intramolecular interaction(s) involving regions within the NTF and CTF prior to undergoing endoproteolysis.

Other studies have indicated that the levels of PS NTF and CTF are regulated by association with limiting cellular factors. For example, in transfected mouse N2a cell lines and in the brains of transgenic mice expressing human PS1, accumulation of human PS1-derived NTF and CTF is accompanied by a compensatory, and highly selective, decrease in the steady-state levels of murine PS1 and PS2 derivatives. This “replacement” occurs by post-translational mechanisms independent of endoproteolysis. Indeed, PS have been shown to form high molecular weight complexes involving β-catenin and likely other proteins (Seeger et al., 1997; Capell et al., 1998; Steiner et al., 1998). Although several laboratories identified PS binding proteins using yeast-two-hybrid methodologies, the influence of these interacting proteins on regulated PS proteolysis and/or the saturable accumulation of PS fragments has not been described.

B. FAD-linked PS1 Mutations Influence APP metabolism The mechanisms by which FAD-linked PS variants cause AD are not fully understood, but several important insights have emerged. The most provocative insight came from studies that demonstrated that FAD-linked PS1 and PS2 variants selectively enhance the production of Aβ42 peptides in transfected mammalian cells, the brains of transgenic mice, and patients with AD (Borchelt et al., 1996; Duff et al., 1996; Scheuner et al., 1996; Tomita et al., 1997; Borchelt et al., 1997; Holcomb et al., 1998). Aβ42 peptides are more fibrillogenic than the shorter Aβ40 peptides, and are more prominent in the amyloid lesions of patients with AD (Jarrett et al., 1993; Iwatsubo et al., 1994; Gravina et al., 1995). Thus, while PS 1 is necessary for the y-secretase cleavage of APP, FAD-linked mutant PS bias this cleavage toward the production of highly amyloidogenic Aβ42 species that foster Aβ deposition in brain. The mechanism(s) by which mutant PS influences the production of Aβ42 peptides are uncertain, but FAD-linked mutant PS proteins appear to cause aberrant gain, rather than loss, of function (Sisodia et al., 1999). Understanding how PS proteins influence the production of Aβ42 peptides is of central importance to AD research.

C. Presenilin-Interacting Proteins

Several lines of evidence indicate that PS-derived NTF and CTF are components of high molecular weight complexes (Seeger et al., 1997; Capell et al., 1998; Yu et al., 1998; Steiner et al., 1998). To identify the components of the PS complexes, several investigators including the present inventors employed yeast two-hybrid assays and candidate protein approaches. These efforts have uncovered interactions between presenilins and several proteins (Thinakaran, 1999). Most notable is the identification of interactions between PS1 and members of a family of arnadillo-related proteins including β-catenin, γ-catenin, δ-catenin, p0001, and neural-specific plakophilin (Yu et al., 1998; Zhou et al., 1997; Murayama et al., 1998; Stahl et al., 1999; Levesque et al., 1999; Tanahashi and Tabira, 1999). Although β-catenin is a multifunctional protein involved in Wnt signal transduction, cell adhesion and tumor progression, the functional significance of PS·β-catenin interaction is unclear. In this regard, there have been apparently contradictory findings linking PS1 expression and β-catenin stability (Zhang et al., 1998; Kang et al., 1999). However, FAD-linked mutant PS1 appears to regulate intracellular trafficking of β-catenin (Nishimura et al., 1999). Although the demonstration of interactions between PS1 and β-catenin is provocative, the influence of β-catenin or any of the other known presenilin interacting proteins on the regulated metabolism of presenilins or the enhanced production of Aβ42 by FAD mutants has not been reported.

D. Presenilins Facilitate Intramembranous Processing of APP, APLP1 and Notch 1

Analysis of APP processing in neurons from mice containing a targeted deletion of PS1 revealed that PS1 is required for γ-secretase cleavage of APP; PS1 deficiency is associated with defects in the secretion of Aβ peptides and intracellular accumulation of APP C-terminal fragments (CTFs) bearing varying extents of the Aβ region (De Strooper et al., 1998; Naruse et al., 1998). Very recently, Wolfe and colleagues presented evidence for two critical aspartate residues within TMs 6 and 7 of PS1 that play an important role in γ-secretase processing of APP (Wolfe et al., 1999); mutation at either of the aspartate residues leads to substantial reductions in Aβ secretion and accumulation of APP CTFs. Regardless of whether PS is a critical co-factor for γ-secretase or itself is the γ-secretase, it is remarkable that FAD-linked PS that harbors independent mutations at multiple TMs and linker domains can specifically influence the generation of Aβ42 peptides, despite the fact that PS1 is required for the production of both Aβ40 and Aβ42. Thus, the connection between FAD-linked PS mutations, its influence on the γ-secretase and enhanced production of Aβ42 is far from clear.

In addition to APP cleavage, it is also apparent that loss of PS1 activity also interferes with intramembranous processing of Notch 1 in Drosophila and mammalian cells (Ye et al., 1999; Struhl and Greenwald, 1999; De Strooper et al., 1999). In PS1-deficient fibroblasts, a constitutively active Notch 1 polypeptide (mNotchAE) is inefficiently processed (De Strooper et al., 1999). This cleavage event is critical for Notch function because it releases the intracellular domain from the membrane; upon release, the intracellular domain is translocated to the nucleus where it activates transcription of Notch target genes. In addition, CTFs derived from the APP homologue, APLP1, also accumulate in PS1^{-/-} neurons (Naruse et al., 1998). Finally, the loss of PS1 expression also affects the biology of other integral membrane glycoproteins, including the receptor tyrosine kinase, TrkB; the rate of oligosaccharide modification and brain derived neurotrophic factor-mediated autophosphorylation of TrkB is severely compromised in PS1^{-/-} neurons (Naruse et al., 1998). In support of these observations, Levitan and Greenwald have demonstrated that in C. elegans, reduced SEL-12 activity results in diminished apical membrane accumulation and signaling of LIN-12/Notch in vulval precursor cells (Levitan and Greenwald, 1998). Since SEL-12 was localized primarily to the ER/Golgi, these findings would be consistent with a role of PS in membrane protein trafficking.

E. Antibiotic Resistance Genes

The present invention provides a novel expression cloning strategy, which utilizes chimeric proteins comprising regions of unstable proteins linked to regions of marker gene products that in some embodiments are exemplified by antibiotic-resistance proteins expressed in stable mammalian brain cells. In one example, this is exemplified by a chimera of the PS1/Ble protein in N2a cells. In other examples, this chimera is further comprised of a visualization marker gene such as a fluorescent protein (FP) marker gene, i.e., FP/PS1/Ble, to aid visualization and fluorescent quantitation of the protein.

The main property of the antibiotic resistance gene product used here is that it provides resistance to the antibiotic at a level that is proportional to the amount of product accumulated in the cell. Thus, if more of the antibiotic resistance gene product is accumulated the cell will be viable in higher concentrations of the antibiotic. In some instances, the antibiotic resistance gene product provides a stoichiometric resistance to the antibiotic by binding at a 1:1 ratio of antibiotic resistance gene product:antibiotic. Thus, when candidate substances increase the accumulation of the unstable protein, and there is an increase in the accumulation of the chimeric unstable protein linked to the antibiotic resistance gene product, there is an increase in the amount of the antibiotic resistance gene product which can be selected at higher concentrations of the antibiotic.

One such example is the bleomycim resistance gene, Ble, that provides resistance to zeocin and other antibiotics of the bleomycin family. Bleomycin resistance (or zeocin resistance) is a dominant selectable marker that is unique in that level of resistance is proportional to the expression/accumulation of the resistant protein (Ble). The sh ble gene of Streptoalloteichus hindustanus confers resistance to bleomycin (or zeocin) and other related antibiotics. Sh ble encodes a stable 14 kDa protein (124 amino acids), Ble, which is non-toxic for cultured mammalian cells. The bleomycin family of antibiotics are glycoproteins that enter the nucleus of cells and cause DNA damage wich leads to cell death. Ble forms high affinity 1:1 complex with belomycin and confers resistance by preventing the antibiotic from reaching the nucleus (Gatignol et al., 1988). Because of the stoichiometric nature of interaction, resistance to bleomycin (zeocin) proportionally correlates with the expression levels of Ble both in prokaryotic and eukaryotic cells (Gatignol et al., 1988). The present inventors have demonstrated that the PS-Ble chimeras of the invention also confer antibiotic resistance to bleomycin and zeocin.

Spontaneous mutations that confer increased resistance to zeocin (or bleomycin) occur at very low frequency (estimated at 6.5×10⁻⁷ per cell per generation in CHO cells) (Akiyama and Kuwano, 1981), and frequently involves increased belomycin hydrolase activity, and to a lesser extent by membrane changes that prevent the antibiotic from entering the cells. In the present invention, when used with the expression cloning method, these false positives will be eliminated during the second round of screening because these mutations will not be recovered in the PCR based retroviral cDNA insert amplification and isolation. Thus, the expression cloning method of the present invention selectively isolates cDNAs that participate in regulating PS accumulation.

Other antibiotic resistance genes that may be used in context with the present invention include those that provide resistance to the following antibiotics: cleomycins, phleomycins, zorbamycins, victomycin, platomycins, tallysomycins, SF 1771, SF 1961 and YA 56, any analogue thereof as well as mixtures of these antibiotics. However, as will be recognized by the skilled artisan, the practice of this invention is not limited by these examples and any antibiotic resistance gene/protein that provides antibiotic resistance that is either stoichiometric or proportional to its expression/accumulation level can be used to construct the chimeric proteins of the invention.

F. Fluorescent Proteins

The present invention provides chimeric proteins comprising regions of unstable proteins linked to fluorescent proteins (FP) as a marker gene, such as the green fluorescent proteins (GFP), yellow fluorescent proteins (YFP), etc., for example, PS-FP. In addition, the chimeric proteins of the invention may additionally comprise another marker gene such as an antibiotic resistance gene and the FP is used here as a visualization marker gene for example, FP/PS1/Ble, to aid visualization and fluorescent quantitation of the protein. The FP's, originally isolated from the jellyfish Aequorea. Victoria (for example, GFP) retain their fluorescent properties when expressed in heterologous cells thereby provides a powerful tool as fluorescent recombinant probes to monitor cellular events or functions (Chalfie et al 1994; Prasher 1995; WO 95/07463).

Several spectral and mutational variants of GFP proteins have since been isolated, for example, the naturally occurring blue-fluorescent variant of GFP (Heim et al. 1994; U.S. Pat. No. 6,172,188, both incorporated herein by reference), the yellow-fluorescent protein variant of GFP (Miller et al., 1999; Weiss, et al.,2001; Majoul, et al., 2001; Laird et al., 2001; Daabrowski et al., 1999), and more recently the red fluorescent protein isolated from the coral Discosoma (Fradkov et al., 2000; Miller et aL, 1999), which allows the use of fluorescent probes having different excitation and emission spectra permitting the simultaneous monitoring of more than one process. GFP proteins provide non-invasive assays which allow detection of cellular events in intact, living cells. The skilled artisan will recognize that the invention is not limited to the fluorescent proteins described and one may use any other spectral or mutational variant or derivative.

Several methods to identify and quantitate cells that are fluorescently tagged with fluorescent gene products are well known in the art and may be used in the context of the present invention. One example is the use of fluorescent activated cell sorting (FACS), flow cytometry or flow microfluorometry provides the means of scanning individual cells for the presence of an a fluorescent protein. The method employs instrumentation that is capable of activating, and detecting the excitation emissions of cells that express a fluorescent marker in a liquid medium. FACS is unique in its ability to provide a rapid, reliable, quantitative, and multiparameter analysis on either living or fixed cells in culture or in vivo. Other methods to measure fluorescent markers are also well known.

G. Screening for Modulators of Protein Accumulation

The present invention provides methods for identifying modulators that lead to the accumulation or hyperaccumulation of unstable proteins as well as other membrane proteins that require associations with other proteins/molecules. These proteins are also referred to as ‘proteins of interest’ and include unstable proteins such as the presenilin proteins, ion channels such as the N-ACh receptor, GABA receptors, glycine receptors, Na⁺, Ca⁺, or K⁺ channels and the like. The screening assays may comprise random screening of large libraries of candidate substances. Alternatively, the assays may be used to focus on particular classes of compounds selected with an eye towards structural attributes that are believed to make them more likely to modulate the levels and/or the accumulation of the proteins of interest. Thus, the screening assays will assay for increases or decreases in levels of proteins of interest in response to a candidate substance.

Alternatively, one may assay for a change in function or activity of the protein. The change may be an increase or a decrease in activity or function of the protein. By function, it is meant that one may assay for any protein related biological activity, such as an increased/decreased enzyme activity, an increased/decreased electrical activity corresponding to increased/decreased levels of ion channels, transcriptional activity measured directly or via promoter assays (CAT assays or luciferase assays), Ca⁺⁺ imaging, cell surface expression of marker proteins, cell survival or cell death, etc.

A modulator is defined here as any substance that alters the accumulation of a protein of interest. As described in the previous sections substances and/or molecules that modulate the levels of proteins may be involved in several pathologies. For example, modulators of presenilin levels are directly implicated in the pathology of Alzheimer's disease.

To identify a modulator that changes the levels of accumulation of an unstable protein, one generally will determine the level of the protein of interest, such as a presenilin, in the presence and absence of the candidate substance. For example, the methods of the invention generally comprise:

a) obtaining a cell or animal expressing a chimeric polypeptide comprising a polypeptide of the unstable protein linked to a marker gene product;

b) exposing the cell or animal to a candidate substance/modulator; and

c) determining any change in a level of the chimeric protein subsequent to exposing the cell or animal with the candidate substance/modulator.

An increase in the level of the unstable protein is readily measurable by the expression of the chimeric polypeptide and can be further quantified using suitable marker gene products. In some embodiments, the marker gene products are fluorescent gene products such as the green fluorescent protein (GFP), yellow fluorescent protein (YFP), and other similar spectral and mutational variants. In other embodiments, the marker gene products are antibiotic resistance gene products that provide a proportionate resistance to increasing concentrations of an antibiotic depending on the level of expression of the antibiotic resistance gene product. For instance, the bleomycin resistance gene product, sh ble, provides a stoichiometric resistance to bleomycin or similar antibiotics by binding to bleomycin at a 1:1 ratio and conferring resistance to a cell expressing sh ble. Therefore, a cell expressing a chimeric protein which is a fusion of an unstable protein and sh ble will be resistant to concentrations of bleomycin that are directly proportional to the levels of the chimeric protein. Hence, a modulator or candidate substance that increases the level of the unstable protein can be easily identified by screening for cells that survive in higher concentrations of bleomycin.

In the cases where the functional change due to the increased accumulation of a protein of interest are measured, the methods comprise;

(a) providing a candidate modulator;

(b) admixing the candidate modulator with a cell, or a suitable experimental animal;

(c) measuring one or more characteristics of the cell or animal in step (c); and

(d) comparing the characteristic measured in step (c) with the characteristic of the cell or animal in the absence of said candidate modulator,

wherein a difference between the measured characteristics indicates that said candidate modulator is, indeed, a modulator of the protein of interest.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

a. Modulators

As used herein the term “candidate substance” refers to any molecule that may potentially increase levels of or enhance activity of the unstable proteins or proteins of interest of the invention. The candidate substance may be a protein or fragment thereof, a small molecule, or even a nucleic acid molecule. It may prove to be the case that the most useful pharmacological compounds will be compounds that are structurally related to the unstable protein. Whatever the structure or the nature of the substances identified by the methods of the present invention one can use the identified substances to further develop compounds for therapeutic uses. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or other compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

b. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing levels of a protein, the ability of a modulator to bind to a target molecule in a specific fashion is strong evidence of a related biological effect such as its ability to interact with and/or stabilize an unstable protein. For example, binding of a molecule to a unstable protein or to a membrane protein may stabilize the protein and prevent it from degradation thereby leading to accumulation of such a proteins. The interaction may be due to steric, allosteric or charge-charge interactions. The unstable protein may be either free in solution, fixed to a support, expressed in or on the surface of a cell. Either the unstable protein or the compound may be labeled, thereby permitting determining of binding. Usually, the unstable protein will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

c. In cyto Assays

The present invention further contemplates the screening of compounds for their ability to modulate levels of unstable proteins in cells. Various cell lines can be utilized for such screening assays, including cells specifically engineered for this purpose. For example, for analyzing compounds that can modulate the levels of the PS proteins, the N2a neuroblastoma cell line provides a good cell line as this cell is known to process PS proteins in the same way that normal cells do. Thus, the N2a cell line provides a bioenvironment that comprises proteins and nucleic acids that regulate PS accumulation and stabilization. These cells can be suitably engineered to express the chimeric constructs of the invention by the methods described herein. In addition, several other cells and cell lines such as African monkey kidney COS cells, Chinese hamster ovary CHO cells, human embryonic kidney 293 cells, human neuroblastoma SH-SY5Y, human neuronal Ntera-2 cells and derivatives, rat pheochromocytoma PC12, NIH 3T3 fibroblasts cells, etc. may be used.

Depending on the assay, cell culture may be required. The cell is examined using any of a number of different physiologic assays. Alternatively, molecular analysis may be performed, for example, looking at protein expression, mRNA expression (including differential display of whole cell or polyA RNA) and others.

d. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter the levels of the unstable proteins are measured, as compared to a similar animal not treated with the candidate substance(s), identifies a modulator. In addition to the levels, one may also measure other characteristics of the unstable protein that may be altered as a result of a increase in the level of the unstable protein. These characteristics may be a change with regard to the function of a particular unstable protein, e.g., enzyme, receptor, hormone, ion channel, or instead a broader indication such as behavior, neurological response, physiological response, pathological response, etc.

The present invention provides methods of screening for a candidate substance that modulates the levels of accumulation of unstable proteins. In these embodiments, the present invention is directed to a method for determining the ability of a candidate substance to decrease the hyperaccumulation of an unstable protein such as a presenilin protein or any amyloid type of protein, generally including the steps of: administering a candidate substance to the animal; and determining the ability of the candidate substance to reduce one or more characteristics of a disease or pathology caused by the hyperaccumulation or symptoms associate with the same. In specific embodiments, the disease or pathology is Alzheimer's disease.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intracerebral, intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

H. Nucleic Acid-Based Expression Systems

1. Vectors

The term “vector” is used to refer to a carrier nucleic acid molecule into which a nucleic acid sequence can be inserted for introduction into a cell where it can be replicated. In the present invention, vectors are used to introduce nucleic acid sequences that correspond to the chimeric protein constructs of the invention into cells or cell lines. In addition, vectors may also be used to introduce candidate nucleic acid modulators of levels of unstable proteins into a cell for screening.

A nucleic acid sequence can be “exogenous”, which means that it is foreign to the cell into which the vector is being introduced or that the sequence is homologous to a sequence in the cell but in a position within the host cell nucleic acid in which the sequence is ordinarily not found. The vectors described in the present invention are predominantly plasmid and viral based. However, other vectors that can be used include cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases RNA molecules are then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Imler et |
| | al., 1987; Weinberger et al., |
| | 1984; Kiledjian et al., 1988; Porton et al.; |
| | 1990 |
| Immunoglobulin | Queen et al., 1983; Picard et al., 1984 |
| Light Chain | |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine | Jaynes et al., 1988; Horlick et al., |
| Kinase (MCK) | 1989; Johnson et al., 1989 |
| Prealbumin | Costa et al., 1988 |
| (Transthyretin) | |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsch et al., 1990 |
| Adhesion Molecule | |
| (NCAM) | |
| α₁-Antitrypsin | Latimer et al., 1990 |
| H2B (TH2B) Histone | Hwang et al., 1990 |
| Mouse and/or | Ripe et al., 1989 |
| Type I Collagen | |
| Glucose-Regulated | Chang et al., 1989 |
| Proteins (GRP94 | |
| and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; Sleigh |
| | et al., 1985; Firak et al., 1986; Herr et al., |
| | 1986; Imbra et al., 1986; Kadesch et al., 1986; |
| | Wang et al., 1986; Ondek et al, 1987; Kuhl et |
| | al., 1987; Schaffner et al., 1988 |
| Polyoma | Swartzendruber et al., 1975; Vasseur et al., |
| | 1980; Katinka et al., 1980, 1981; Tyndell et |
| | al., 1981; Dandolo et al., 1983; de Villiers et |
| | al., 1984; Hen et al., 1986; Satake et al., 1988; |
| | Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; Cel- |
| | ander et al., 1987; Thiesen et al., 1988; Cel- |
| | ander et al., 1988; Choi et al., 1988; Reisman |
| | et al., 1989 |
| Papilloma Virus | Campo et al., 1983; Lusky et al., 1983; Span- |
| | didos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla et al., 1986; Jameel et al., 1986; Shaul |
| | et al., 1987; Spandau et al., 1988; Vannice et |
| | al., 1988 |
| Human | Muesing et al., 1987; Hauber et al., 1988; |
| Immunodeficiency | Jakobovits et al., 1988; Feng et al., 1988; Tak- |
| Virus | ebe et al., 1988; Rosen et al., 1988; Berkhout et |
| | al., 1989; Laspia et al., 1989; Sharp et al., |
| | 1989; Braddock et al., 1989 |
| Cytomegalovirus (CMV) | Weber et al., 1984; Boshart et al., 1985; |
| | Foecking et al., 1986 |
| Gibbon Ape | Holbrook et al., 1987; Quinn et al., 1989 |
| Leukemia Virus | |

TABLE 2
Inducible Elements

| Element | Inducer | References |
|---|---|---|
| MT II | Phorbol Ester | Palmiter et al., 1982; Haslinger |
| | (TFA) Heavy | et al., 1985; Searle et al., 1985; |
| | metals | Stuart et al., 1985; Imagawa |
| | | et al., 1987, Karin et al., 1987; |
| | | Angel et al., 1987b; McNeall |
| | | et al., 1989 |
| MMTV (mouse | Glucocorticoids | Huang et al., 1981; Lee et al., |
| mammary tumor | | 1981; Majors et al., 1983; |
| virus) | | Chandler et al., 1983; Ponta |
| | | et al., 1985; Sakai et al, 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | E1A, SV40 Large | Taylor et al., 1989, 1990a, |
| | T Antigen | 1990b |
| Proliferin | Phorbol Ester- | Mordacq et al., 1989 |
| | TPA | |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (olio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct expressing the chimeric proteins of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). The cDNA expression library component of the present invention may be comprised as a viral vector that encode various proteins some of which will be molecules that regulate the levels of accumulation of the PS proteins or any other protein of interest. Although the invention describes retroviral cDNA expression libraries, other viral vectors can also be used in conjunction with the invention. Non-limiting examples of various viral vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

A nucleic acid may be introduced into a cell or cell line using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the cDNA library expression system of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are a preferred embodiment as vectors in context of the cDNA library expression system of the present invention due to their ability to integrate their genes into the host genome, transferring a large amount of foreign genetic material, infecting a broad spectrum of species and cell types and of being packaged in special cell-lines (Miller, 1992).

In order to construct a retroviral cDNA expression system vector, a nucleic acid (e.g., one encoding a protein that modulates the accumulation of PS) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific. Thus, lentiviral vectors are especially preferred retroviral cDNA expression library vectors in the present invention.

4. Other Viral Vectors

Other viral vectors may be employed as cDNA expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et aL., 1989).

2. Vector Delivery and Cell Transformation

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al, 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al, 1980; Kaneda et al., 1989; Kato et al, 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by desiccation/inhibition-mediated DNA uptake (Potrykus et aL, 1985), and any combination of such methods. Through the application of techniques such as these, organelle(s), cell(s), tissue(s) or organism(s) may be stably or transiently transformed.

a. Injection

In certain embodiments, a nucleic acid may be delivered to an organelle, a cell, a tissue or an organism via one or more injections (i.e., a needle injection). Further embodiments of the present invention include the introduction of a nucleic acid by direct microinjection. Direct microinjection has been used to introduce nucleic acid constructs into Xenopus oocytes (Harland and Weintraub, 1985).

b. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

c. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

d. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

e. Sonication Loading

Additional embodiments of the present invention include the introduction of a nucleic acid by direct sonic loading. LTK- fibroblasts have been transfected with the thymidine kinase gene by sonication loading (Fechheimer et al., 1987).

f. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et aL, 1991). In yet further embodiments, a liposome may be complexed or employed in conjunction with both HVJ and HMG-1. In other embodiments, a delivery vehicle may comprise a ligand and a liposome.

g. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et aL, 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987).

h. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et aL, 1987). There are a wide variety of microprojectile bombardment techniques known in the art, many of which are applicable to the invention.

Microprojectile bombardment may be used to transform various cell(s), tissue(s) or organism(s). One or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. In preferred embodiments of the present invention, the host cell is a eukaryotic cell which further is a mammalian cell. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. The host cells here are transformed with the chimeric proteins of the invention and in some aspects these are further co-transfected with cDNA's expressing candidate genes that can regulate accumulation of unstable proteins. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result.

Examples of eukaryotic host cells for replication and/or expression of a vector include, but are not limited to, Phoenix, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

I. Proteins, Polypeptides, and Peptides

a. Chimeric Proteins

A chimeric or fusion proteins is a specialized kind of protein variant that is an insertional variant. This molecule generally has all or a substantial portion of the native molecule, linked at the N- or C-terminus or in even at other parts of the protein, to all or a portion of a second polypeptide. In the present invention, chimeric proteins have been generated that comprise regions/portions of the unstable protein and regions of a marker gene product that can be identified using either a screening assay, a functional assay or a fluorescence measuring method. For example, chimeras comprising a region of a presenilin protein linked to an antibiotic resistance gene product such as the bleomycin resistance gene product, Ble, are described. In many of these examples the Ble protein encoding nucleic acid is fused to hydrophilic loop regions of the presenilin proteins which were found to be dispensable with respect to PS function in a series of deletion analysis experiments. In other examples, regions of green fluorescent proteins (GFP) or yellow fluorescent proteins (YFP) are fused to unstable proteins to form a chimeric protein such as Unstable-Protein/GFP. Some of the GFP chimeras are N-terminal chimeras. Almost any type of fusion/chimeric protein may be prepared as long as the chimeric protein retains the normal processing and metabolism of the unstable protein. Other useful chimeras include linking of functional domains, such as active sites from enzymes.

b. Protein purification

The present invention also contemplates isolating and purifying proteins or polypeptides that interact with the unstable proteins and thereby regulate their accumulation and stability. For example one may isolate and purify regulators of PS protein accumulation or hyperaccumulation. The latter contribute to the pathology of AD. One can also purify proteins that associate with and stabilize ion channels and other membrane receptor proteins such as the N-AChR, voltage gated ion-channels etc. The purification of such proteins is contemplated by their ability to interact with the unstable proteins. Thus, the present invention provides purified, and in preferred embodiments, substantially purified, proteins, polypeptides, or peptides. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

Although preferred for use in certain embodiments, there is no general requirement that the protein, polypeptide, or peptide always be isolated/ be recovered in their most purified state. Indeed, it is contemplated that less substantially purified protein, polypeptide or peptide, which are nonetheless enriched in the desired protein compositions, relative to the natural state, will have utility in certain embodiments. Methods exhibiting a lower degree of relative purification may have advantages in total recovery of protein product, or in maintaining the activity of an expressed protein.

J. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Generation of Cell Lines

In cells transfected with unstable proteins a minor fraction of the unstable protein is processed into stable derivatives. Examples of unstable proteins are polytopic membrane proteins (i.e., multi-pass membrane proteins) with complex structure and/or “co-factors” such as ligand-gated ion channels (nicotinic acetylcholine receptors, GABA receptors, glycine receptors), and voltage gated ion channels (voltage-gated Na⁺ channels, K⁺ channels, and Ca²⁺0 channels); and PS proteins. For example, in transfected cells overexpressing PS, the vast majority of the synthetic PS polypeptides are rapidly degraded. Hence, stable cell lines were generated to assess the metabolism of the deletion mutants. These cell lines may further comprise one or more FAD mutations. The N2a cell line Swe. 10, which expresses human APP harboring the “Swedish” mutation is one of the cell lines used. These cells are particularly useful as they secrete easily-detectable levels of Aβ. This allows one to analyze PS polypeptide metabolism and also allows the measurement of Aβ production.

Two methods were used to generate stable cells expressing PS polypeptide. In one method, N2a Swe.10 cells were transfected with pCDNA3.1 (Invitrogen) expression plasmids and independent stable clones expressing the deletion mutants were established; alternatively, cDNAs were cloned into a bicistronic vector (pIREHygro, purchased from Clontech) and hygromycin-resistant stable pools of cells were established. In the second method, as the antibiotic selection marker is translated from a bicistronic transcript using an internal ribosomal entry (IRE) site located 3′ to the PS1 coding sequence, all hygromycin resistant cells express PS1. pIRE-transfected pools of cells were also shown to be suitable for assessing FAD-mutation mediated elevations in the Aβ42 levels.

### EXAMPLE 2

### Functional Characterization of PS1 Chimeric Polypeptides

It is known that Presenilins (PS1 and PS2) are required for the intramembraneous cleavage of amyloid precursor protein (APP) and Notchl. To assess whether the chimeric PS1 polypeptides of the invention were functional APP and Notch processing was analyzed. PS1- fibroblasts were transfected with expression plasmids encoding chimeric PS1 molecules, along with a cDNA encoding APP or Notch1. Processing of APP was monitored by the secretion of beta-amyloid (Aβ). Notch processing was monitored by the generation of Notch intracellular domain (NICD). Since PS1^{-/-} fibroblasts completely lack PS1, they do not produce either Aβ or NICD. Transfection with wild-type PS1 “rescued” this deficiency and both Aβ and NICD were produced. The chimeric PS polypeptides of the invention, including PS1-Sh ble, PS-FP as well as the YFP-PS1-Sh ble, were equally efficient in rescuing Aβ and NICD production in transfected PS1^{-/-} fibroblasts demonstrating that the chimeric PS polypeptides are fully functional. In addition, the expression of PS1-Sh ble and the YFP-PS-Sh ble chimeric polypeptides also confers resistance to zeocin/bleomycin.

Nucleic acids encoding YFP-PS1-Sh ble and YFP-PS1-Sh ble bearing the Alzheimer's disease-linked linked M146L mutation were constructed and stably transfected into Swe.10 cells (which are stably transfected N2a cells that express human APP bearing Alzheimer's disease-linked “Swedish” mutation; these cells secrete high levels of Aβ40 and Aβ42), to analyze the expression of the corresponding chimeric polypeptide. The expression and proteolysis of the chimeric polypeptides in stable YFP-PS1-Sh ble and YFP-PS1-Sh ble/M146L cells were compared with the expression of endogenous presenilin in N2a cells by western blotting. The N2a cells expressed the endogenous stable PS derivatives, i.e., the NTF product, as did the YFP-PS1-Sh ble and YFP-PS1-Sh ble/M146L cells, which expressed the chimeric YFP-PS1 NTF showing normal expression and proteolytic processing of the chimeric polypeptide. The chimera also replaces the endogenous PS1 NTF product in YFP-PS1-Sh ble and YFP-PS1-Sh ble/M146L cells. The YFP-PS1-Sh ble chimera was also functionally characterized and was found to support Notch cleavage in an assay that measured the production of NICD. Furthermore, YFP-PS1-Sh ble/M146L also expressed higher levels of Aβ42 as compared to YFP-PS1-Sh ble cells, showing the chimeric presenilin is functional in selectively elevating the levels of Aβ42, as it has been previously reported in N2a cells expressing full-length PS1 M146L.

### EXAMPLE 3

### Intramolecular Associations Between PS Transmembrane Domains

PS1 undergoes endoproteolytic processing in vivo to generate stable (t_{½˜}24 h) NTF and CTF. Co-immunoprecipitation and in situ cross-linking analyses of both cultured cells as well as intact brain tissue demonstrated that NTF and CTF derived from endoproteolytic processing of PS1 remain associated (Suzuki et al., 1994). Other studies also showed that transgene-derived human NTF fails to associate with endogenous mouse PS1 CTF (Tomita et al., 1999). PS proteins are known to form heteromeric assemblies and migrate as high molecular weight complexes on size exclusion columns and velocity density gradients (Seeger et al., 1997; Capell et al., 1998; Steiner et al., 1998). However, it was shown that cells co-expressing PS1 and PS2 do not form heteromeric PS1/PS2 assemblies.

To characterize other intramolecular associations between PS transmembrane domains and to identify and characterize proteins that regulate PS stability and accumulation several experiments were performed.

Saturable Accumulation of PS1 and PS2 Derivatives.

In mouse N2a cell lines expressing human PS1, despite the presence of high levels of full-length PS1 polypeptide, the accumulation of human derivatives is restricted to levels of mouse derivatives present in untransfected cells, indicating that the cleaved PS fragments can not be overproduced. For example, cell lines expressing low and high levels of human PS2 full-length precursors were compared. PS2 expression was analyzed by Western blot using αPS2Loop antiserum, which reacts preferentially with human PS2. Overexpression of full-length PS2 did not lead to appreciable increases in the levels of PS2 CTF in the cell line that has high expression as compared with the cell line with the low expression documenting saturable accumulation of PS2 CTF. Furthermore, the accumulation of endogenous PS1 NTF and CTF is compromised by high level expression of PS2. These results demonstrate that accumulation of PS derivatives is saturable and that levels of PS1 and PS2 derivatives are coordinately regulated.

Saturable Accumulation of PS Derivatives is Regulated by Post-Translational Mechanisms.

One explanation for the overexpressed human PS1/PS2 replacing the endogenous murine PS1/PS2 is that transcripts encoding murine PS1/PS2 are diminished. However, murine PS1 and PS2 mRNA were readily detected in all stable “PS1 lines” and “PS2 lines”. As controls, the steady-state levels of β-tubulin and APP were examined. These studies demonstrate that the steady-state levels of murine PS1 and PS2 mRNA did not changed in cells that overexpress human PS1 or PS2 indicating the role of post-translational mechanisms.

Diminution of Murine PS1 and PS2 is a Selective Effect of Human PS1 or PS2 Overexpression. To confirm that overexpression of human PS1 or PS2 selectively compromises the steady-state accumulation of murine PS1/PS2, the expression of other resident proteins was examined in the ER, a compartment in which the majority of PS1 and PS2 accumulate (Cook et al., 1996; Kovacs et al., 1996; Walter et al., 1996; De Strooper et al., 1997). The steady-state levels of calnexin, a membrane-bound protein that transiently interacts with a variety of newly-synthesized membrane-bound and secretory proteins (Bergeron et al., 1994), and GRP78/BiP, a lumenal ER-resident protein that is induced by accumulation of misfolded ER proteins (Kozutsumi et al., 1988; Watowich and Morimoto, 1988) remained unchanged in stable of PS1 or PS2 cells. Moreover, overexpression of other membrane-bound proteins did not influence the accumulated levels of PS derivatives.

Model for PS1/PS2 Processing in Cells Overexpressing Human PS1.

The results presented herein are consistent with a model presented in FIG. 3. Nascent PS1/PS2 polypeptides are stabilized and targeted to the cleavage pathway by successful interaction with limiting cellular factor(s). In transfected cells and transgenic mice, overexpressed human PS1 competes with endogenous PS1 and PS2 for these interactions. The “excess” PS1 and PS2 polypeptides that are not targeted for the endoproteolytic pathway are rapidly degraded (Ratovitski et al., 1997; Podlisny et al., 1997; Thinakaran et al., 1997), whereas the processed derivatives are turned over with half-lives of 24 h (Thinakaran et al., 1997).

### EXAMPLE 4

### Role of Hydrophilic “Loop” Domain of PS1 and PS2

Protein secondary structure predictions and topological studies have revealed that PS contain 8 transmembrane (TM) domains and 3 stretches of extra-membranous domains longer than 25 amino acids: the N-terminal domain, the C-terminal domain, and a cytoplasmic domain located between TMs 6 and 7 (Doan et al., 1996; Li and Greenwald, 1996; Li and Greenwald, 1998; Lehmann et al., 1997; De Strooper et al., 1997). The studies described in this example are designed to gain more insights on the stretch of hydrophilic residues located within the loop domain. The loop domain of PS (PS1 aa 262-380; the largest cytoplasmic domain of PS) has been the focus several investigations. The sites of regulated endoproteolysis (PS1 aa 292-298) as well as caspase-mediated cleavage (PS1 aa 345) are located within this domain. The loop domain connects TMs 6 and 7, which harbor the pair of aspartate residues that are critical for several biological activities of PS (Wolfe et al., 1999; Steiner et al., 1999). Finally, yeast two-hybrid assays and other methods identified binding sites within the loop domain for several proteins: a family of armadillo-related proteins including β-catenin (Yu et al., 1998; Zhou et al., 1997; Murayama et al., 1998; Stahl et al., 1999; Levesque et al., 1999; Tanahashi and Tabira, 1999; Tesco et al., 1998); APC filarnin, an actin binding protein (Zhang et al., 1998); calcium binding proteins including calmyrin (Stabler et al., 1999) and sorcin; cytoplasmic linker protein termed CLIP-170 (Johnsingh et al., 2000) etc. These findings indicate potentially important role(s) for the loop domain in the functions of PS1 and PS2.

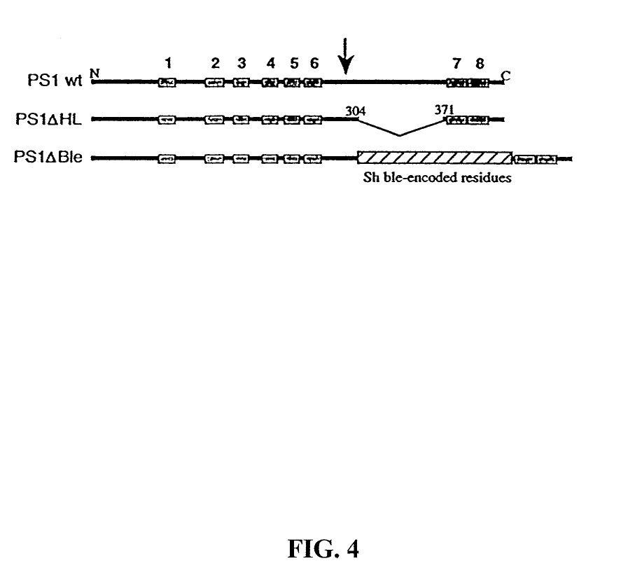

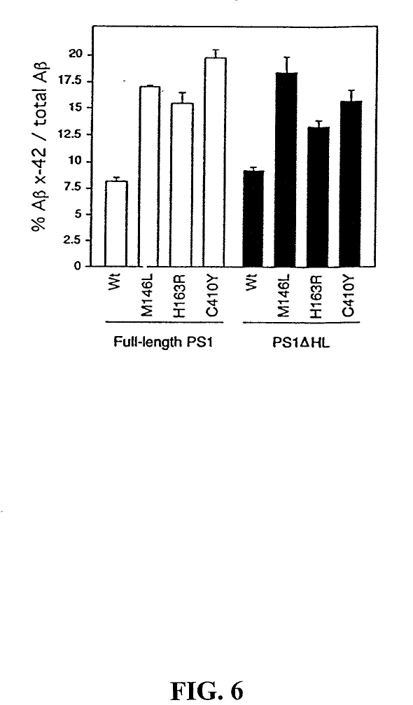

In stably transfected cell lines, a deletion mutant PS1ΔHL (FIG. 4), which lacks a stretch of 68 hydrophilic amino acids of the loop domain undergoes regulated endoproteolytic cleavage similar to wild type PS1, to generate NTF and CTF_{ΔHL}. Expression PS1ΔHL harboring FAD-linked mutations in stable N2a cell lines caused selective increase in the production of the highly fibrillogenic Aβ42 peptides (FIG. 6). These results indicate that in vitro the HL domain is not required for the normal metabolism of PS, and the elevated production of AΔ42 peptides by FAD-linked mutant PS molecules in vitro. However, several issues remain unresolved including: the in vivo role of the interacting proteins that bind to PS HL domain; the role of PS1 HL domain in Aβ42 overproduction and Aβ deposition in the mammalian brain; and the role of PS1 HL domain in mammalian embryonic development. Because in vitro studies cannot address these important questions relating to the biological functions of PS in vivo, the present inventors contemplate addressing these issues in transgenic mice. The inventors envision that such studies will reveal whether the PS1 HL domain is required for mammalian embryonic development and Aβ accumulation/deposition in mouse brain.

Generation of PS1 Polypeptide Lacking the Hydrophilic Loop Domain.

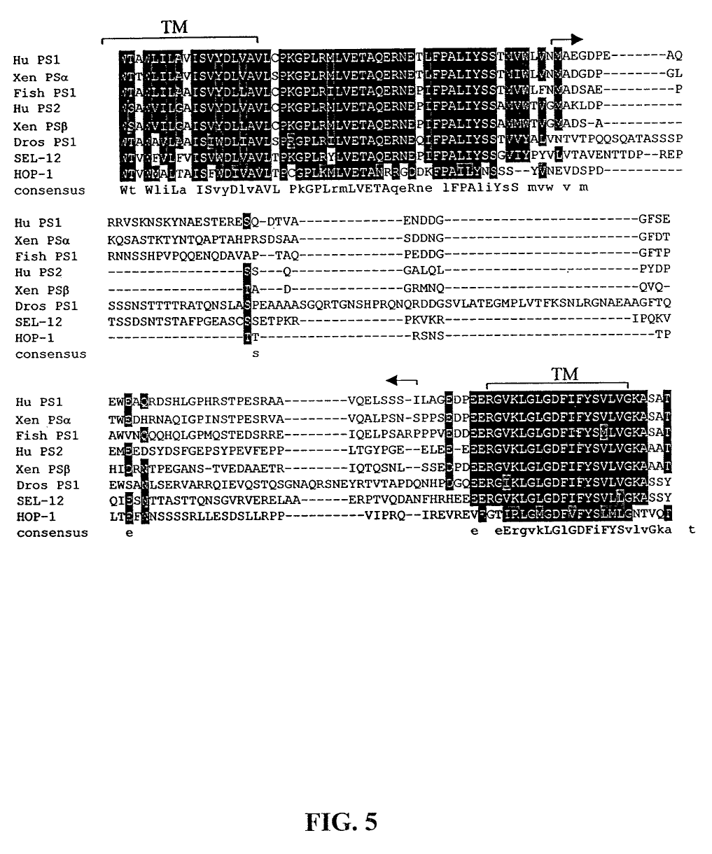

To investigate whether the non-conserved HL region of PS plays an essential role in PS1 metabolism, a cDNA that encodes a human PS1 polypeptide without the non-conserved HL region (Glu 304-Gly 371; marked by arrows in FIG. 5), termed PS1ΔHL can be generated. The non-conserved region within the loop domain connecting TMs 6 and 7 of PS was determined by multiple sequence analysis of PS1 and PS homologues using ClustalW (FIG. 5). A 345-base pair DNA fragment that encodes PS1 amino acids 195-303 and contains PflM1 and BbsI sites at the 5′ and 3′ end, respectively, was amplified by PCR using the primers 5′-CGCTACATTACTGTTGCACTCC-3′ (SEQ ID NO:5) and 5′-GGCCTCTGGGTCTTCCGGGTCTCCTTCTGC-3′ (SEQ ID NO:6). The resulting PCR product was digested with PflM1 and BbsI, and ligated to plasmid pBSPS1 (Thinakaran et al., 1996) digested with PflM1 and BbsI. After sequencing the amplified region, the PS1ΔHL coding sequence was cloned into pCDNA3.1 expression vector to generate PS1ΔHL. In the resulting was region deleted. The site of regulated endoproteolytic cleavage of PS1 (Met 292) (Podlisny et al., 1997; Steiner et al, 1999) is present in PS1ΔHL, but the site of caspase cleavage (Asp 345) is been deleted.

Metabolism of PS1ΔHL Polypeptide and Accumulation of Derivatives.

The metabolism of PS1ΔHL was also examined in independent N2a clones expressing PS1ΔHL. Antibody PS1NT, (Thinakaran et aL, 1998), reacted with the “full-length” PS1ΔHL and ˜30 kDa PS1ΔHL-derived human NTF, which exhibited slightly accelerated migration on gels compared to endogenous mouse NTF and co-migrated with full-length human PS1-derived NTF. In each of the stable PS1ΔHL clones, αPS1Loop (which fails to react with PS1ΔHL) only detected a weak signal for the mouse PS1-derived CTFs as compared with cells transfected with empty vector, indicating diminished accumulation of endogenous murine PS1 derivatives. Western blots using PS-C3, an antibody raised against the COOH-terminus of PS1 revealed the accumulation of the 18 kDa PS1 CTF, and 9 kDa CTF_{ΔHL} derived from PS1AHL. Finally, PS1_{NT} antiserum coimmunoprecipitates NTF and CTF_{ΔHL} from detergent extracts of stable N2a cells expressing PS1ΔHL. These results demonstrate that the deletion of the HL domain of PS1 does not affect the regulated endoproteolytic processing of PS1, saturable accumulation of PS1 fragments, or the association of NTF and CTF.

The HL Domain is Dispensable for FAD-Linked PS-Mediated Elevation of Aβ42 Production.

The role of HL domain of PS1 in the FAD mutation-mediated increase in the production of Aβ42 were then investigated. cDNAs that encode PS1ΔHL polypeptides harboring the FAD-linked missense mutation M146L, H163R, or C410Y were generated. To measure Aβ production, Wt or mutant PS1ΔHL cDNAs were cotransfected into COS cells along with a cDNA that encodes APPswe. ELISA quantification revealed 2-fold increase in Aβ42/total Aβ ratio in conditioned media collected from cells transfected with mutant full-length PS 1 (% of Aβ42/total Aβ for PS1 Wt was 8.04±0.38 versus PS1 mutants, 17.28±0.73; P<0.0001). Aβ42 ratio was not significantly different between cells transfected with Wt PS1 and PS1AHL (PS1 was 8.04±0.38 versus PS1ΔHL, 9.03±0.35; P=0.959), indicating that deletion of the HL domain, by itself, did not influence the production of Aβ42 (FIG. 6). Notably, Aβ42 ratio was elevated 1.7-fold by the expression PS1AHL harboring FAD-linked mutations M146L, H163R, and C410Y (PS1ΔHL Wt was 9.03±0.35 versus PS1ΔHL mutants, 15.7±0.91; P=0.0026).

Generation of APP/PS2ΔL Double Stable N2a Cell Lines.

In order to examine the influence of HL domain on Aβ40 and Aβ42 production in stably transfected cells, stable N2a cells that coexpressed APPswe, and PS2 or PS2ΔHL polypeptides were generated. Swe.10, a stable N2a cell line which expresses human APP695 harboring the “Swedish” double mutation, was transfected with empty vector, full-length PS2, or PS2ΔHL cDNA cloned in a bicistronic vector, and stable pools of hygromycin resistant cells were derived. Western blotting with PS²NT, a PS2 NH₂-terminal antibody revealed the presence of 30 kDa PS2 NTF. As expected from clonal lines, each of the stable pools synthesized similar levels of APP, quantified by 10 min pulse labeling using [³⁵S]methionine followed by immunoprecipitation and phosphorimaging analysis. Secreted Aβ40 and Aβ42 in media conditioned by the double stable pools were analyzed by fractionation on bicine/urea gels (Wiltfang et al., 1997) followed by immunoblotting with monoclonal antibody 26D6. Consistent with the results using transient transfection of COS cells, FAD mutations in PS2 or PS2ΔHL caused marked increase in the levels of Aβ42 in stable N2a cells.

Deletion of the HL Domain Does not Influence Aβ Production in Double Stable N2a Cell Lines.

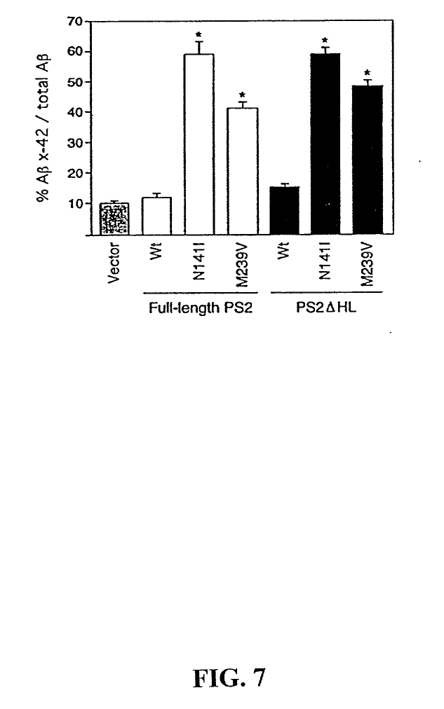

To quantify the levels of Aβ40 and Aβ42 a two-site ELISA was performed. No significant differences in Aβ40 secreted by PS2ΔHL cells as compared with full-length PS2 cells or cells transfected with an empty vector were seen. The only significant difference in the amounts of secreted Aβ40 was observed in PS1 N1411 cells (P=0.035, N1411 versus vector transfected cells). The small increase in the amounts of Aβ42 secreted by PS2ΔHL cells as compared with PS1 Wt cells did not reach statistical significance by ANOVA. Furthermore, the percentage of Aβ42/total Aβ was also not significantly different between PS2Wt and PS2AHL cells (11.68±1.32 and 15.04±0.82, respectively; P=0.97) (FIG. 7). As expected from previous studies (Tomita et al., 1997), the amounts of secreted Aβ42 were significantly higher in FAD-linked mutant cells (7.68-and 5.74-fold more in PS2 N141I and PS2 M239V, respectively; P<0.0001), compared to PS2 Wt cells. Consistent with immunoblotting results, the amounts of secreted Aβ42 were also significantly higher from mutant PS2AHL cells (7.72- and 5.41-fold more in N141I and M239V, respectively; P<0.0001), compared to PS2ΔHL cells (FIG. 7). Collectively, these results demonstrate that the deletion of the HL domain of PS1 or PS2 has little influence on the levels of secreted Aβ. Furthermore, FAD-linked mutations elevated the levels of secreted Aβ42 to similar levels in cells expressing the full-length or PS2ΔHL molecules.

### EXAMPLE 5

### Deletion Analysis

A series of deletion analysis of PS proteins was performed to identify the domains involved in PS1 endoproteolysis, stability and function.

Endoproteolysis.

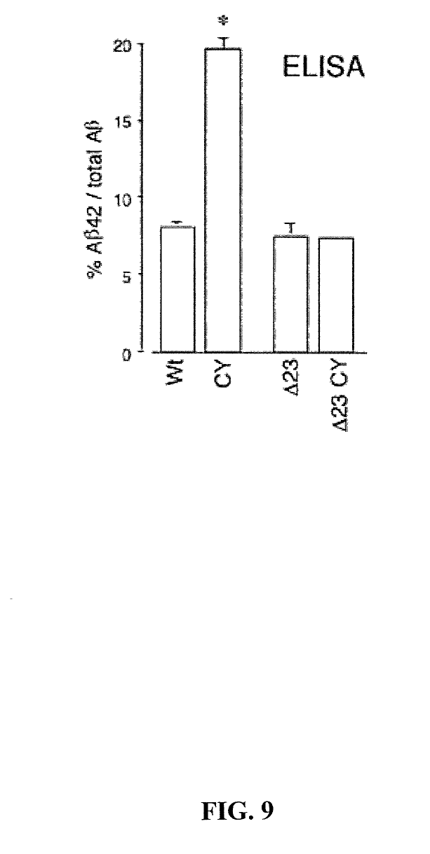

Deletion analysis was performed. Of particular interest here, deletion of transmembrane domains 2 and 3 affects PS1 endoproteolysis as described below. Stable N2a cell lines were generated by transfecting Swe.10 cells with a expression plasmid that encodes PS1 lacking the transmembrane domains 2 and 3 (refered to as Δ2,3 cells). Western blot analysis of lysates from Δ2,3 cells using PS1 antibodies revealed that unlike the full-length PS1 polypeptide, which undergoes endoproteolysis to generate stable NTF and CTF, the Δ2,3 polypeptide accumulates as holoproteins. Furthermore, overexpression of Δ2,3 PS1 failed to interfere with the accumulation of endogenous PS1 NTF and CTF, indicating lack of “replacement” typically observed when full-lentgh human PS1 is overexpressed in N2a cells. FIG. 9 demonstrates the analysis of Aβ42 secretion by the expression of Δ2,3. COS cells were trasnsfected with WtΔ2,3 or Δ2,3 bearing the Alzheimer's disease-linked C410Y mutation (Δ2,3C410Y). Conditioned media collected from transfected cells were analyzed by sandwich ELISA. The results show that unlike full-length PS1 harboring the C410Y substitution, expression of Δ2,3C410Y failed to elevate the levels of Aβ42. Together with the Western blot analysis these results show that Δ2,3 polypeptides are not endoproteolytically processed and not functional in elevating Aβ42 production. Furthermore, protein stability analysis performed by incubating cells with cycloheximide to inhibit protein translation showed that Δ2,3 polypeptides are unstable and have very short half-lives.

Stability and Function.

The PS1 polypeptide bearing the deletion of transmembrane domains 2 and 3, referred to as the ΔM2,3 polypeptide, was found to be unstable, failed to elevate the levels of Aβ42 and also failed to support Notch cleavage. In addition, the ΔM2,3-Sh ble chimeric polypeptide was also unstable and failed to replace endogenous PS1.

Zeocin Resistance.

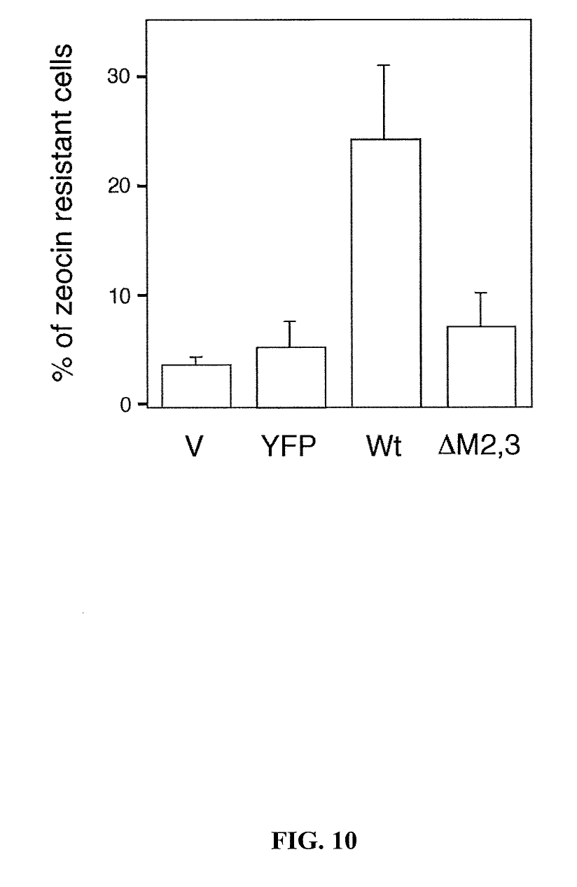

The inventors also investigated the ability of the chimeric ΔM2,3-Sh ble polypeptide to confer resistance to zeocin. For this, cells were co-transfected with either the PS1-Sh ble as controls or with PS1ΔM2,3-Sh ble, and a hygromycin resistant vector. The transfected cells were screened for both zeocin resistance as well as hygromycin resistance and the percentage of zeocin resistant cells were calculated by the formula: Zeocin Resistant cells/Hygromycin Resistant cells×100. Only the stable chimeric PS1 polypeptides conferred antibiotic resistance as measured by a cell viability assay in the presence of zeocin (FIG. 10). Cells expressing the ΔM2,3-Sh ble chimeric polypeptide, which was unstable by biochemical analysis, were not resistant to the antibiotic zeocin (FIG. 10).

### EXAMPLE 6

### Zeocin Toxicity Studies

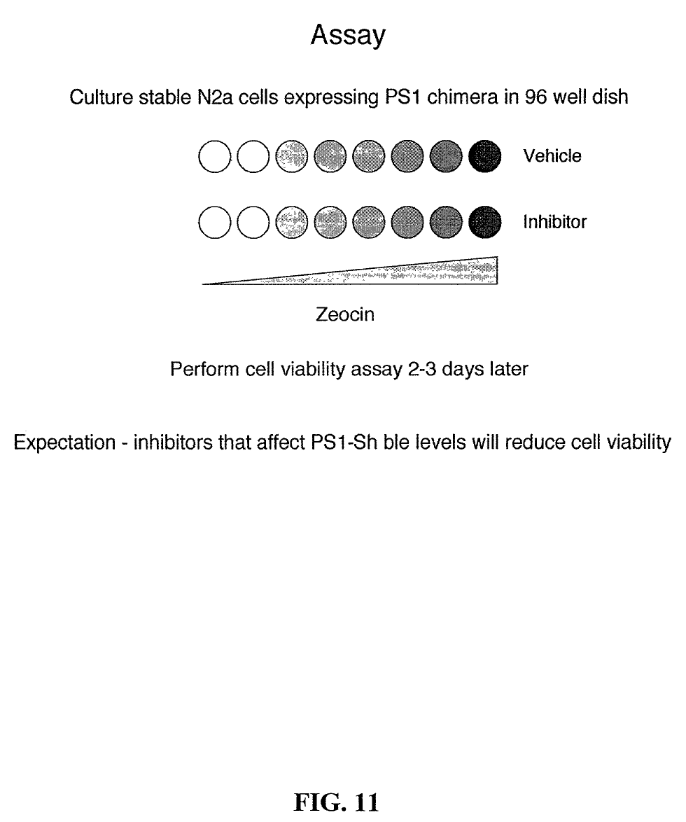

Cultured stable N2a cells expressing chimeric PS1 polypeptides were tested for cell viability in the presence of increasing concentrations of zeocin and in the presence and absence of known presenilin inhibitors in multi-well formats (FIG. 11). The inhibitor tested, namely L-686,485 has been previously reported to bind to PS1 and PS2 (Shearman et al., 2000), therefore it was expected that exposure of cells to this inhibitor might exacerbate cell death observed in the presence of high concentrations of zeocin. In these assays, cell viability can be assayed by commercially available cell viability ELISA kits.

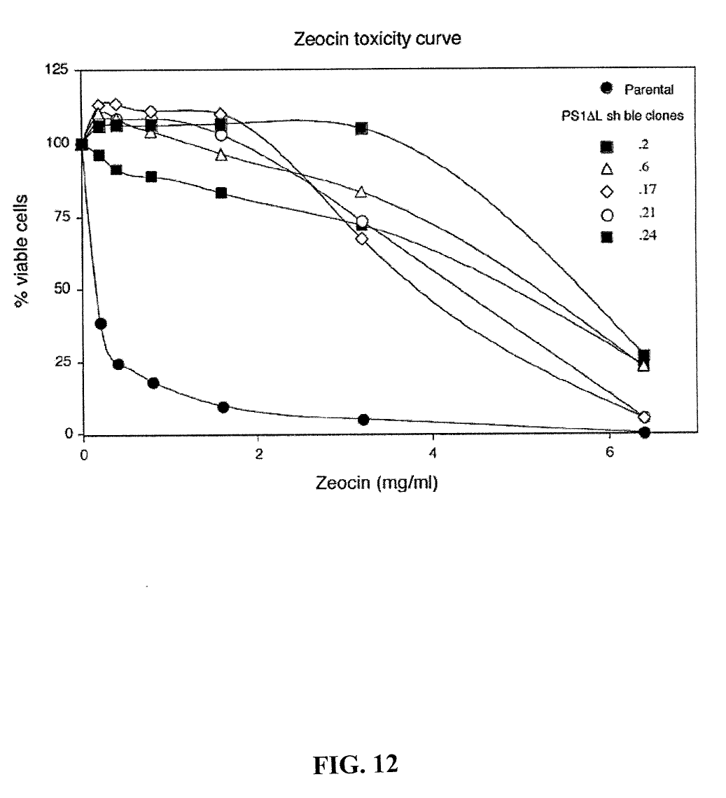

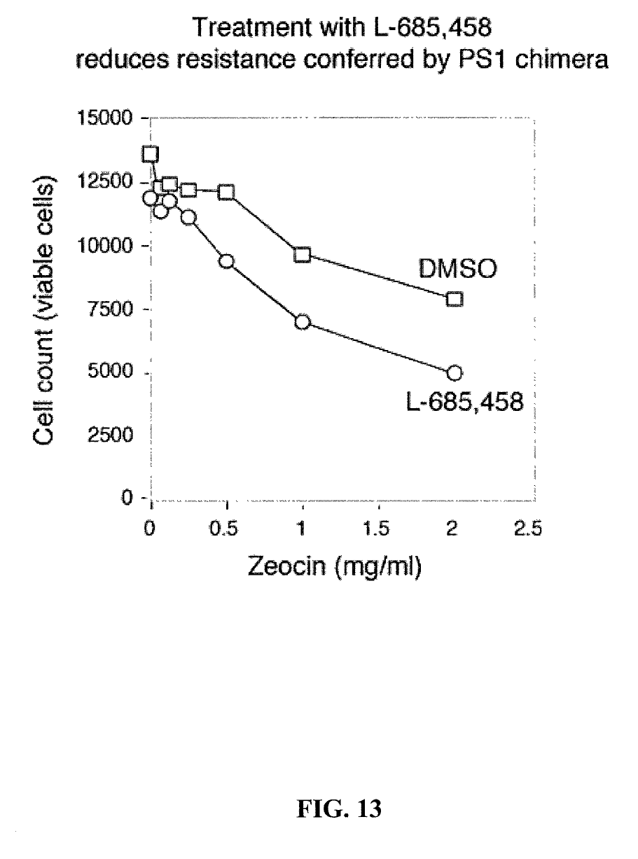

Stable N2a cells expressing PS1 chimeric proteins or the parental N2a cell line were plated at a density of 6.25×10³ cells/well into 96-well tissue culture dishes in a volume of 90 l/well. Serial dilutions of Zeocin were added (2 mg/ml to 0.0625 mg/ml final concentration) in a volume of 10 l/well. In some experiments the γ-secretase inhibitor, L-686,485 (purchased from Bachem) was added at a final concentration of 1 μM to one set of wells. The cells were incubated from 2 to 3 days at 37° C. Then 10 μl of Cell Proliferation Reagent WST-1 (Roche Biochemical) was added to each well. The plate was shaken for 30 sec. then placed back in the 37° C. incubator. The plate was read at 450 nm after 30 min., 1 h, 2 h and 3h in a Benchmark Plus Absorbance Plate Reader (BioRad). These experiments demonstrate that cells expressing PS1 chimera are viable at zeocin concentrations of 0-4 mg/ml whereas the parental cells are significantly less viable even at the lowest zeocin concentration tested (0.2 mg/ml) (FIG. 12). Treatment with inhibitors of PS such as L-685,458 reduced the resistance to zeocin conferred by the PS1 chimera and the cells were viable at 0-2 mg/ml of zeocin (FIG. 13).

The advantages of this screening method is that the assay is a simple cell viability assay that is easily performed. The toxicity of the inhibitors does not influence the assay as cells expressing chimeras are resistant to high concentrations of zeocin and can still be screened at lower concentrations of zeocin in the presence of the inhibitors of unstable proteins. As will be appreciated by one of skill in the art, although the screening methods described here are for PS proteins the assay is adaptable for any protein of interest.

### EXAMPLE 7

### Expression Cloning Method

To identify proteins that participate in the regulation of PS accumulation, an expression cloning method was designed based on resistance to the antibiotic bleomycin (an alternate commercial product is called zeocin). Bleomycin is a glycopeptide antibiotic that binds to DNA and introduces strand scission and release of thymidine residues (Muller and Zahn, 1977). The sh ble gene of Streptoalloteichus hindustanus confers resistance to bleomycin (zeocin) and related antibiotics. Sh ble encodes a stable 14 kDa protein (124 amino acids), Ble, which is non-toxic for cultured mammalian cells. Ble forms high affinity 1:1 complex with belomycin and confers resistance by preventing the antibiotic from reaching the nucleus (Gatignol et al., 1988). Due to the stoichiometric nature of interaction, resistance to bleomycin (zeocin) proportionally correlates with the expression levels of Ble both in prokaryotic and eukaryotic cells (Gatignol et al., 1988). A chimeric PS1-Ble fusion protein was created in a novel screening assay utilizing the drug-resistance properties of Ble. A deletion of 68 residues of the PS1 hydrophilic loop (HL) domain (amino acids 304-371) has no effect on the metabolism and in vitro functions of PS1; see FIG. 6 and FIG. 7).

To generate PS1ΔBle, Sh ble coding sequences were introduced in frame at the HL deletion junction (aa 304/771) of PS1ΔHL. Stable N2a cell lines expressing PS1ΔBle were generated and the ability of this chimera to undergo endoproteolysis, similar to PS1 lacking HL (PS1ΔHL) was confirmed. The sh ble gene was fused in frame to alcohol dehydrogenase gene (3′, 5′ or internal fusions) or lacZ gene (5′ and 3′ fusions); in all cases, expression of the resulting chimeric protein conferred resistance to bleomycin. Since Ble binds belomycin or zeocin at 1:1 stoichiometry, the level of resistance strictly correlates with the level of accumulated full-length PS3ΔBle and PS1ΔBleCTF.

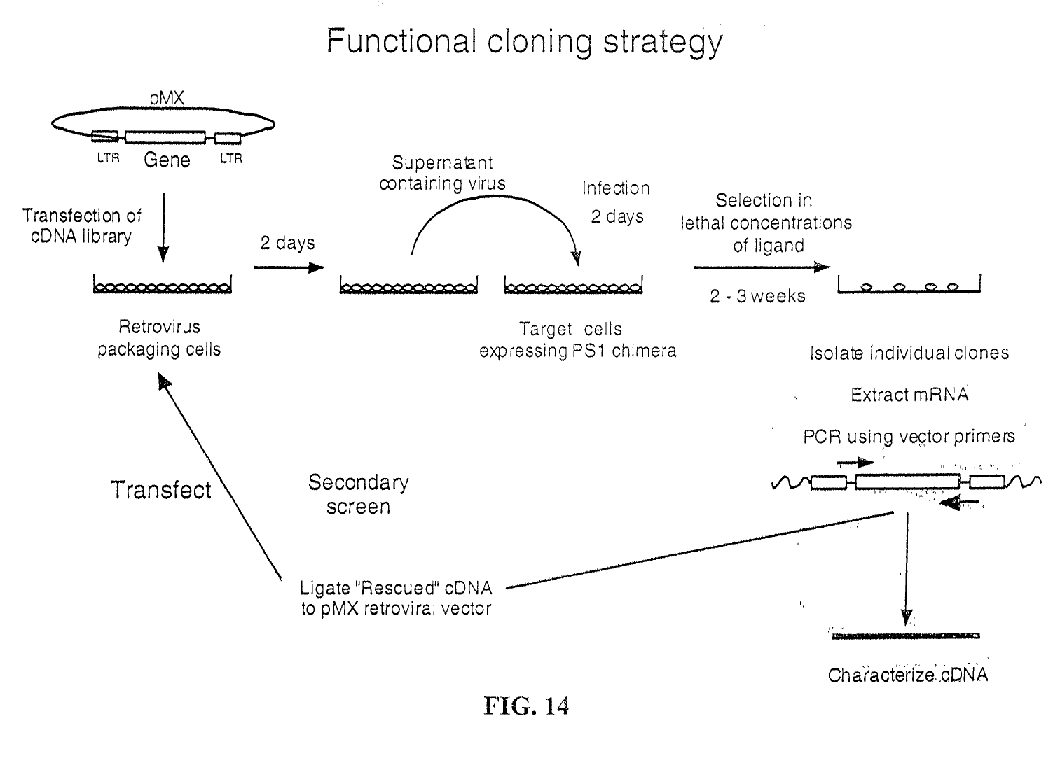

The cDNA library-based expression cloning method using a retroviral vector based cDNA library is outlined in FIG. 14. For the screen, a cell line that expresses very low levels of the PS1ΔBle protein is chosen so that the chimera is largely present as cleaved PS1 NTF and PS1ΔBleCTF. Thus, transgene-derived expression of a putative protein that facilitates increased accumulation of PS1ΔBle confers increased bleomycin (Zeocin) resistance to the target cell. An aliquot of a commonly available cDNA library (5 μg DNA) can then be transfected into human 293-based Phoenix retroviral packaging cells (Hitoshi et al., 1998) and viral supernatant transferred to target N2a PS Able cells to allow infection. After allowing infection/transgene expression for 2-3 days, the infected population of cells are subject to antibiotic selection in 1-2 mg/ml zeocin (5-10 times greater than the concentration normally used for selecting stable clones). At this higher concentration, only cells that accumulate several fold higher levels of PS1ΔBle relative to parental PS1ΔBle cells (that are resistant to 0.2 mg/ml), form colonies. After selecting such resistant clones, the cDNA corresponding to putative candidate proteins that increase/modulate/regulate/stabilize the presenilins can be “rescued” by PCR amplification of genomic DNA isolated from either pooled resistant clones (if more than 25 survived zeocin selection) or individually picked clones (if less than 25 clones), using vector primers and religated to the original retroviral vector pMX. Rescued cDNAs in pMX vector can then be used for a second round of packaging/infection/antibiotic selection. If overexpression of a particular cDNA caused increased accumulation of PS1ΔBle, and therefore, an increased resistance to zeocin, in a clone, the cDNA rescued from the clone will confer increased resistance to the parental PS1ΔBle cells during the rescreening. Surviving clones from the second round of screening can then be individually picked for further analysis.

### Expression Construct and Characterization of PS1ΔBle.

Sequences encoding Ble were generated by PCR using pZeoSV, a cloning vector that harbors the sh ble gene, using the following primers: BleF-5′ CCTGAAGACCCAGAGATGGCCAAGTTGACCAGTGCC 3′ (SEQ ID NO:1), BleR 5′ CCTCTCTGGGTCTTCGTCCTGCTCCTCGGCCACGAAG 3′ (SEQ ID NO:2). After digestion with BbsI, the DNA fragment was subcloned into the BbsI site in plasmid PS1ΔHL. The resulting cDNA encoded a chimeric PS polypeptide PS1ΔBle, in which the HL domain is replaced with bleomycin resistant protein residues. Independent stable cell lines can then be generated by transfecting N2a cells with PS1ΔBle cDNA. Metabolism of the chimeric PS protein can be characterized by assessing endoproteolysis, regulated accumulation of derivatives, and replacement of murine PS fragments. Moreover, clones expressing both low and high levels of chimeric proteins can be used determine their sensitivity to the antibiotic zeocin. Cells expressing low levels of PSlABle (equal to or less than endogenous PS fragments) would only be resistant to ˜200 μg/ml of zeocin a concentration routinely used to select stable N2a cells by transfecting plasmids containing sh ble selection marker.

Retroviral Methods.

One can generate recombinant retroviruses by transient transfection of a suitable retroviral cDNA library into Phoenix packaging cells using calcium phosphate method. Retroviral supernatant can be harvested 24 h after transfection and filtered to remove cellular debris. Infection of target N2a PS1ΔBle cells can be accomplished by adding the viral supernatant in the presence of Polybrene (4 μg/ml) for 48 h. At this time, the medium is replaced with medium containing 1 or 2 mg/ml zeocin. The sensitivity of target cells to the high concentrations of zeocin can be established by parallel infections of parental N2a PS1ΔBle cells with a retroviral vector containing sh ble gene under the control of a strong CMV enhancer/promoter (positive control) and the retroviral vector containing an unrelated cDNA insert—lacZ (negative control). Resistant clones can then be individually picked or pooled (depending on the number of colonies) after about 3 weeks of selection.

Genomic DNA can then be isolated as described previously (Sambrook et al., 1989), from clones selected after the second round of screening. Integrated retroviral cDNAs can be recovered by PCRs using for example the pMX primer pair, 5′-CCACCGCCCTCAAAGTAGACG (SEQ ID NO:3), and 5′-CCAACTTAATCGCCTTGCAGCA (SEQ ID NO:4). The products can then be cloned into pCDNA vectors for transfection studies.

Characterization of Potential Candidates.

The candidate proteins that are isolated using the expression cloning method described above can then be further characterized. Rescued cDNA encoding potential candidate proteins (or the corresponding full-length cDNA, if available from other sources) can be used to generate stable cell lines to confirm increased accumulation of endogenous PS1 derivatives in untransfected N2a cell lines. Potential direct physical interaction between candidate proteins and PS1 full-length polypeptide or fragments can be addressed by co-immunoprecipitation and cross-linking studies as described above. The outcome of hyperaccumulation of wild-type and mutant PS1 polypeptides on APP processing and Aβ production can be further characterized in cell lines expressing FAD-linked mutant PS1 polypeptide.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and method and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Akiyama and Kuwano, “Isolation and preliminary characterization of bleomycin-resistant mutants from Chinese hamster ovary cells,” J Cell Physiol, 107:147-153, 1981.

Almendro, Bellon, Rius, Lastres, Langa, Corbi, Bemabeu, “Cloning of the human platelet endothelial cell adhesion molecule-l promoter and its tissue-specific expression. Structural and functional characterization,” J. Immunol., 157(12):5411-5421, 1996.

Angel, Bauman, Stein, Dellus, Rahmsdorf, and Herrlich, “12-0-tetradecanoyl-phorbol-13-acetate Induction of the Human Collagenase Gene is Mediated by an Inducible Enhancer Element Located in the 5′ Flanking Region,” Mol. Cell. Biol., 7:2256, 1987a.

Angel, Imagawa, Chiu, Stein, Imbra, Rahmsdorf, Jonat, Herrlich, and Karin, “Phorbol Ester-Inducible Genes Contain a Common cis Element Recognized by a TPA-Modulated Trans-acting Factor,” Cell, 49:729, 1987b Ausubel, Brent, Kingston, Moore, Seidman, Smith, Struhl, In: Current Protocols in Molecular Biology, John, Wiley & Sons, Inc, New York, 1994.

Baichwal and Sugden, “Vectors for gene transfer derived from animal DNA viruses: Transient and stable expression of transferred genes,” In: Gene transfer, Kucherlapati (ed.), NY Plenum Press, 117-148, 1986.

Banerji et al., “Expression of a Beta-Globin Gene is Enhanced by Remote SV40 DNA Sequences,” Cell, 27:299, 1981.

Banerji, Olson, and Schaffner, “A lymphocyte-specific cellular enhancer is located downstream of the joining region in immunoglobulin heavy-chain genes,” Cell, 35:729, 1983.

Bergeron, Brenner, Thomas, Williams, “Calnexin: a membrane-bound chaperone of the endoplasmic reticulum,” Trends Biochem Sci, 19:124-128, 1994.

Berkhout, Silverman, and Jeang, “Tat Trans-activates the Human Immunodeficiency Virus Through a Nascent RNA Target,” Cell, 59:273, 1989.

Blanar, Baldwin, Flavell, and Sharp, “A gamma-interferon-induced factor that binds the interferon response sequence of the MHC class I gene, H-2 Kb,” EMBO J., 8:1139, 1989.

Blomer, Naldini, Kafri, Trono, Verma, Gage, “Highly efficient and sustained gene transfer in adult neurons with a lentivirus vector,” J. Virol., 71(9):6641-6649, 1997.

Bodine and Ley, “An enhancer element lies 3′ to the human a gamma globin gene,” EMBO J., 6:2997, 1987.

Borchelt, Davis, Fischer, Lee, Slunt, Ratovitsky, Regard, Copeland, Jenkins, Sisodia, “A vector for expressing foreign genes in the brains and hearts of transgenic mice,” Genet Anal, 13:159-163, 1996.

Borchelt, Ratovitski, van Lare, Lee, Gonzales, Jenkins, Copeland, Price, Sisodia, “Accelerated amyloid deposition in the brains of transgenic mice coexpressing mutant presenilin 1 and amyloid precursor proteins,” Neuron, 19:939-945, 1997.

Borchelt, Thinakaran, Eckman, Lee, Davenport, Ratovitsky, Prada, Kim, Seekins, Yager, Slunt, Wang, Seeger, Levey, Gandy, Copeland, Jenkins, Price, Younkin, Sisodia, “Familial Alzheimer's disease-linked presenilin 1 variants elevate Abetal-42/1-40 ratio in vitro and in vivo,” Neuron, 17:1005-1013, 1996.

Boshart, Weber, Jahn, Dorsch-Hasler, Fleckenstein, and Schaffner, “A very strong enhancer is located upstream of an immediate early gene of human cytomegalovirus,” Cell, 41:521, 1985.

Bosze, Thiesen, and Charnay, “A transcriptional enhancer with specificity for erythroid cells is located in the long terminal repeat of the friend murine leukemia virus,” EMBO J., 5:1615, 1986.

Braddock, Chambers, Wilson, Esnouf, Adams, Kingsman, and Kingsman, “HIV-I Tat activates presynthesized RNA in the nucleus,” Cell, 58:269, 1989.

Bulla and Siddiqui, “The hepatitis B virus enhancer modulates transcription of the hepatitis B virus surface-antigen gene from an internal location,” J. Virol., 62:1437, 1986.

Campbell and Villarreal, “Functional analysis of the individual enhancer core sequences of polyoma virus: cell-specific uncoupling of DNA replication from transcription,” Mol. Cell. Biol., 8:1993, 1988.

Campere and Tilghman, “Postnatal repression of the alpha-fetoprotein gene is enhancer independent,” Genes and Dev., 3:537, 1989.

Campo, Spandidos, Lang, Wilkie, “Transcriptional control signals in the genome of bovine papilloma virus type 1,” Nature, 303:77, 1983.

Capell, Grunberg, Pesold, Diehlmann, Citron, Nixon, Beyreuther, Selkoe, Haass, “The proteolytic fragments of the Alzheimer's disease-associated presenilin-1 form heterodimers and occur as a 100-150-kDa molecular mass complex,” J. Biol. Chem., 273:3205-3211, 1998.

Carbonelli et al. “A plasmid vector for isolation of strong promoters in E. coli,” FEMS Microbiol Lett. 177(1):75-82, 1999.

Celander and Haseltine, “Glucocorticoid Regulation of Murine Leukemia Virus Transcription Elements is Specified by Determinants Within the Viral Enhancer Region,” J. Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Chalfie, Tu, Euskirchen, Ward, Prasher, “Green fluorescent protein as a marker for gene expression,” Science, 263(5148):802-805, 1994.

Chandler et al., “RNA splicing specificity determined by the coordinated action of RNA recognition motifs in SR proteins,” Proc Natl Acad Sci USA. 94(8):3596-3601, 1997.

Chandler, Maler, and Yamamoto, “DNA Sequences Bound Specifically by Glucocorticoid Receptor in vitro Render a Heterlogous Promoter Hormone Responsive in vivo,” Cell, 33:489, 1983.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative Regulation of the Thyroid-Stimulating Hormone Alpha Gene by Thyroid Hormone: Receptor Interaction Adjacent to the TATA Box,” Proc Natl. Acad Sci. U.S.A., 86:9114, 1989.

Chen and Okayama, “High-efficiency transformation of mammalian cells by plasmid DNA,” Mol. Cell. Biol. 7:2745-2752, 1987.

Choi, Chen, Kriegler, and Roninson, “An altered pattern of cross-resistance in multi-drug-resistant human cells results from spontaneous mutations in the mdr-1 (p-glycoprotein) gene,” Cell, 53:519, 1988.

Cocea, “Duplication of a region in the multiple cloning site of a plasmid vector to enhance cloning-mediated addition of restriction sites to a DNA fragment,” Biotechniques, 23:814-816, 1997.

Cohen, Walter, and Levinson, “A Repetitive Sequence Element 3′ of the Human c-Ha-ras1 Gene Has Enhancer Activity,” J. Cell. Physiol., 5:75, 1987.

Cook, Sung, Golde, Felsenstein, Wojczyk, Tanzi, Trojanowski, Lee, Doms, “Expression and analysis of presenilin 1 in a human neuronal system: localization in cell bodies and dendrites,” Proc. Natl. Acad. Sci. USA, 93:9223-9228, 1996.

Costa, Lai, Grayson, and Darnell, “The Cell-Specific Enhancer of the Mouse Transthyretin (Prealbumin) Gene Binds a Common Factor at One Site and a Liver-Specific Factor(s) at Two Other Sites,” Mol. Cell. Biol., 8:81, 1988.

Cotten et al., “High efficiency receptor-mediated delivery of small and large (48 kilobase) gene constructs using the endosome disruption activity of defective or inactivated adenovirus particles,” Proc. Natl. Acad. Sci. USA, 89:6094-6098, 1992.

Coupar et al., “A general method for the construction of recombinant vaccinia virus expressing multiple foreign genes,” Gene, 68:1-10, 1988.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745, 1987.

Culotta and Hamer, “Fine Mapping of a Mouse Metallothionein Gene Metal-Response Element,” Mol. Cell. Biol., 9:1376, 1989.

Curiel, “Gene transfer mediated by adenovirus-polylysine DNA complexes,” In: Viruses in Human Gene Therapy, J.-M. H. Vos (Ed.), Carolina Academic Press, Durham, N.C., pp 179-212,1994.

Daabrowski S, Brillowska A, Kur J. Use of the green fluorescent protein variant (YFP) to monitor MetArg human proinsulin production in Escherichia coli. Protein Expr Purif. 1999 July; 16(2):315-23.

Dandolo, Blangy, and Kamen, “Regulation of Polyma Virus Transcription in Murine Embryonal Carcinoma Cells,” J. Virology, 47:55, 1983.

Davis, Naruse, Chen, Eckman, Younkin, Price, Borchelt, Sisodia, Wong, “An Alzheimer's disease-linked PS1 variant rescues the developmental abnormalities of PS1-deficient embryos,” Neuron, 20, 603-609, 1998.

De Strooper, Annaert, Cupers, Saftig, Craessaerts, Mumm, Schroeter, Schrijvers, Wolfe, Ray, Goate, Kopan, “A presenilin-1-dependent gamma-secretase-like protease mediates release of Notch intracellular domain,” Nature, 398:518-522, 1999.

De Strooper, B., Saftig, P., Craessaerts, K., Vanderstichele, H., Guhde, G., Annaert, W., Von Figura, K., and Van Leuven, F. (1998) Deficiency of presenilin-1 inhibits the normal cleavage of amyloid precursor protein. Nature, 391, 387-390.

De Strooper, Beullens, Contreras, Levesque, Craessaerts, Cordell, Moechars, Bollen, Fraser, George-Hyslop, Van Leuven, “Phosphorylation, subcellular localization, and membrane orientation of the Alzheimer's disease-associated presenilins,” J. Biol. Chem., 272:3590-3598, 1997.

De Villiers, Schaffner, Tyndall, Lupton, and Kamen, “Polyoma Virus DNA Replication Requires an Enhancer,” Nature, 312:242, 1984.

Deschamps, Meijlink, and Verma, “Identification of a Transcriptional Enhancer Element Upstream From the Proto-Oncogene Fos,” Science, 230:1174, 1985.

Doan, Thinakaran, Borchelt, Slunt, Ratovitsky, Podlisny, Selkoe, Seeger, Gandy, Price, Sisodia, “Protein topology of presenilin 1,” Neuron, 17:1023-1030, 1996.

Duff, Eckman, Zehr, Yu, Prada, Perez-tur, Hutton, Buee, Harigaya, Yager, Morgan, Gordon, Holcomb, Refolo, Zenk, Hardy, Younkin, “Increased amyloid-beta42(43) in brains of mice expressing mutant presenilin 1,” Nature, 383:710-713, 1996.

Edbrooke, Burt, Cheshire, and Woo, “Identification of cis-acting sequences responsible for phorbol ester induction of human serum amyloid a gene expression via a nuclear-factor-kappa β-like transcription factor,” Mo!. Cell. Biol., 9:1908, 1989.

Edlund, Walker, Barr, and Rutter, “Cell-specific expression of the rat insulin gene:

evidence for role of two distinct 5′ flanking elements,” Science, 230:912, 1985.

EPO 0273085

Fechheimer, Boylan, Parker, Sisken, Patel and Zimmer, “Transfection of mammalian cells with plasmid DNA by scrape loading and sonication loading,” Proc Nat'l.

Acad. Sci. USA 84:8463-8467, 1987.

Feng and Holland, “HIV-1 Tat Trans-Activation Requires the Loop Sequence Within Tar,” Nature, 334:6178, 1988.

Firak and Subramanian, “Minimal Transcription Enhancer of Simian Virus 40 is a 74-Base-Pair Sequence that Has Interacting Domains,” Mol. Cell. Biol., 6:3667, 1986.

Foecking and Hofstetter, “Powerful and Versatile Enhancer-Promoter Unit for Mammalian Expression Vectors,” Gene, 45(1):101-105, 1986.

Fradkov, Chen, Ding, Barsova, Matz, Lukyanov, “Novel fluorescent protein from Discosoma coral and its mutants possesses a unique far-red fluorescence,” FEBS Lett., 479(3):127-30, 2000.

Fraley, Fomari, Kaplan, “Entrapment of a bacterial plasmid in phospholipid vesicles:potential for gene transfer,” Proc Nat'l. Acad. Sci. USA 76:3348-3352, 1979.

Friedmann, “Progress toward human gene therapy,” Science, 244:1275-1281, 1989.

Fujita, Shibuya, Hotta, Yamanishi, and Taniguchi, “Interferon-Beta Gene Regulation:

Tandemly Repeated Sequences of a Synthetic 6-bp Oligomer Function as a Virus-Inducible Enhancer,” Cell, 49:357, 1987.

Gatignol, Durand, Tiraby, “Bleomycin resistance conferred by a drug-binding protein,” FEBS Lett., 230:171-175, 1988.

Ghosh and Bachhawat, “Targeting of liposomes to hepatocytes,” In: Liver Diseases, Targeted Diagnosis and Therapy Using Specific Receptors and Ligands, Wu and Wu (Eds.), Marcel Dekker, New York, pp 87-104, 1991.

Gilles, Morris, Oi, and Tonegawa, “A tissue-specific transcription enhancer element is located in the major intron of a rearranged immunoglobulin heavy-chain gene,” Cell, 33:717, 1983.

Gloss, Bernard, Seedorf, and Klock, “The Upstream Regulatory Region of the Human Papilloma Virus-16 Contains an E2 Protein-Independent Enhancer Which is Specific for Cervical Carcinoma Cells and Regulated by Glucocorticoid Hormones,” EMBO J., 6:3735, 1987.

Godbout, Ingram, and Tilghman, “Fine-Structure Mapping of the Three Mouse Alpha-Fetoprotein Gene Enhancers,” Mol. Cell. Biol., 8:1169, 1988.

Goodbourn and Maniatis, “Overlapping Positive and Negative Regulatory Domains of the Human β-Interferon Gene,” Proc. Natl. Acad. Sci. USA, 85:1447, 1988.

Goodbourn, Burstein, and Maniatis, “The Human Beta-Interferon Gene Enhancer is Under Negative Control,” Cell, 45:601, 1986.

Gopal, “Gene transfer method for transient gene expression, stable transformation, and cotransformation of suspension cell cultures,” Mol. Cell. Biol. 5:1188-1190, 1985.

Graham and Van Der Eb, “A new technique for the assay of infectivity of human adenovirus 5 DNA,” Virology 52:456-467, 1973.

Gravina, Ho, Eckman, Long, Otvos Jr., Younkin, Suzuki, Younkin, “Amyloid beta protein (A beta) in Alzheimer's disease brain. Biochemical and immunocytochemical analysis with antibodies specific for forms ending at A beta 40 or A beta 42(43),” J. Biol. Chem., 270:7013-7016, 1995.

Greene, Bohnlein, and Ballard, “HIV-1, and Normal T-Cell Growth: Transcriptional Strategies and Surprises,” Immunology Today, 10:272, 1989.

Grosschedl and Baltimore, “Cell-Type Specificity of Immunoglobulin Gene Expression is Regulated by at Least Three DNA Sequence Elements,” Cell, 41:885, 1985.

Grunberg, Walter, Loetscher, Deuschle, Jacobsen, Haass, “Alzheimer's disease associated presenilin-1 holoprotein and its 18-20 kDa C-terminal fragment are death substrates for proteases of the caspase family,” Biochemistry, 37:2263-2270, 1998.

Grunhaus and Horwitz, “Adenovirus as cloning vector,” Seminar in Virology, 3:237-252, 1992.

Harland and Weintraub, “Translation of mRNA injected into Xenopus oocytes is specifically inhibited by antisense RNA,” J. Cell Biol. 101:1094-1099, 1985.

Haslinger and Karin, “Upstream Promoter Element of the Human Metallothionein-II Gene Can Act Like an Enhancer Element,” Proc Natl. Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Heim, Prasher, Tsien, “Wavelength mutations and posttranslational autoxidation of green fluorescent protein,” Proc Natl Acad Sci USA, 91(26):12501-4, 1994.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 E1A Products,” Nature, 321:249, 1986.

Hensel, Meichle, Pfizenmaier, and Kronke, “PMA-Responsive 5′ Flanking Sequences of the Human TNF Gene,” Lymphokine Res., 8:347, 1989.

Herr and Clarke, “The SV40 Enhancer is Composed of Multiple Functional Elements That Can Compensate for One Another,” Cell, 45:461, 1986.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuif, and Gordis, “Identification of Positive and Negative Regulatory Elements Governing Cell-Type-Specific Expression of the Neural-Cell-Adhesion-Molecule Gene,” Mol. Cell. Biol., 10:1959, 1990.

Hitoshi, Lorens, Kitada, Fisher, LaBarge, Ring, Francke, Reed, Kinoshita, Nolan, “Toso, a cell surface, specific regulator of Fas-induced apoptosis in T cells,” Immunity, 8:461-471, 1998.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Holcomb, Gordon, McGowan, Yu, Benkovic, Jantzen, Wright, Saad, Mueller, Morgan, Sanders, Zehr, O'Campo, Hardy, Prada, Eckman, Younkin, Hsiao, Duff, “Accelerated Alzheimer-type phenotype in transgenic mice carrying both mutant amyloid precursor protein and presenilin 1 transgenes,” Nature Med., 4:97-100, 1998.

Horlick and Benfield, “The Upstream Muscle-Specific Enhancer of the Rat Muscle Creatine Kinase Gene is Composed of Multiple Elements,” Mol. Cell. Biol., 9:2396, 1989.

Horwich et al. “Synthesis of hepadenovirus particles that contain replication-defective duck hepatitis B virus genomes in cultured HuH7 cells,” J. Virol., 64:642-650, 1990.

Huang, Ostrowski, Berard, and Hagar, “Glucocorticoid regulation of the ha-musv p21 gene conferred by sequences from mouse mammary tumor virus,” Cell, 27:245, 1981.

Hug, Costas, Staeheli, Aebi, and Weissmann, “Organization of the Murine Mx Gene and Characterization of its Interferon- and Virus-Inducible Promoter,” Mol. Cell. Biol., 8:3065, 1988.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Imagawa, Chiu, and Karin, “Transcription Factor Aβ-2 Mediates Induction by Two Different Signal-Transduction Pathways: Protein Kinase C and cAMP,” Cell, 51:251, 1987.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Inouye et al., “Up-promoter mutations in the lpp gene of Escherichia coli,” Nucl. Acids Res., 13:3101-3109, 1985.

Iwatsubo, Odaka, Suzuki, Mizusawa, Nukina, Ihara, “Visualization of A beta 42(43) and A beta 40 in senile plaques with end-specific A beta monoclonals: evidence that an initially deposited species is A beta 42(43),” Neuron, 13:45-53, 1994.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell Biol., 6:710, 1986.

Jarrett, Berger, Lansbury Jr., “The C-terminus of the beta protein is critical in amyloidogenesis,” Ann N YAcad Sci, 695:144-148, 1993.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johnsingh, Johnston, Merz, Xu, Kotula, Jacobsen, TezaPSIdis, “Altered binding of mutated presenilin with cytoskeleton-interacting proteins(1),” FEBS Lett., 465, 53-58, 2000.

Johnson, Wold, and Hauschka, “Muscle creatine kinase sequence elements regulating skeletal and cardiac muscle expression in transgenic mice,” Mol. Cell. Biol., 9:3393, 1989.

Kadesch and Berg, “Effects of the Position of the Simian Virus 40 Enhancer on Expression of Multiple Transcription Units in a Single Plasmid,” Mol. Cell. Biol., 6:2593, 1986.

Kaeppler et al., Plant Cell Reports 9: 415-418, 1990.

Kaneda et al., “Increased expression of DNA cointroduced with nuclear protein in adult rat liver,” Science, 243:375-378, 1989.

Kang, Soriano, Frosch, Collins, Naruse, Sisodia, Leibowitz, Levine, Koo, “Presenilin 1 facilitates the constitutive turnover of beta-catenin: differential activity of Alzheimer's disease-linked PS1 mutants in the beta-catenin-signaling pathway,” J Neurosci., 19:4229-4237, 1999.

Karin, Haslinger, Heguy, Dietlin, and Cooke, “Metal-Responsive Elements Act as Positive Modulators of Human Metallothionein-IIA Enhancer Activity,” Mol. Cell. Biol., 7:606, 1987.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato et al., “Expression of hepatitis β virus surface antigen in adult rat liver. Co-introduction of DNA and nuclear protein by a simplified liposome method,” J Biol Chem., 266(6):3361-3364, 1991.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kelleher and Vos, “Long-term episomal gene delivery in human lymphoid cells using human and avian adenoviral-assisted transfection,” Biotechniques, 17(6):1110-1117, 1994.

Kiledjian, Su, Kadesch, “Identification and characterization of two functional domains within the murine heavy-chain enhancer,” Mol. Cell. Biol., 8:145, 1988.

Kim, Pettingell, Hallmark, Moir, Wasco, Tanzi, “Endoproteolytic cleavage and proteasomal degradation of presenilin 2 in transfected cells,” J. Biol. Chem., 272:11006-11010, 1997.

Kim, Pettingell, Jung, Kovacs, Tanzi, “Alternative Cleavage of Alzheimer-Associated Presenilins During Apoptosis by a Caspase-3 Family Protease,” Science, 277:373-376, 1997.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein et al., “High-velocity microprojectiles for delivering nucleic acids into living cells,” Nature, 327:70-73, 1987.

Koch, Benoist, and Mathis, “Anatomy of a new β-cell-specific enhancer,” Mol. Cell. Biol., 9:303, 1989.

Kovacs, Fausett, Page, Kim, Moir, Merriam, Hollister, Hallmark, Mancini, Felsenstein, Hyman, Tanzi, Wasco, “Alzheimer-associated presenilins 1 and 2: neuronal expression in brain and localization to intracellular membranes in mammalian cells,” Nature Med., 2:224-229, 1996.

Kozutsumi, Segal, Normington, Gething, Sambrook, “The presence of malfolded proteins in the endoplasmic reticulum signals the induction of glucose-regulated proteins,” Nature, 332:462-464, 1988.

Kraus, Woltje, Schonwetter, Hollt, “Alternative promoter usage and tissue specific expression of the mouse somatostatin receptor 2 gene,” FEBS Lett, 428(3):165-170,1998.

Kriegler and Botchan, “A retrovirus LTR contains a new type of eukaryotic regulatory element,” In: Eukaryotic Viral Vectors, Gluzman (ed.), Cold Spring Harbor, Cold Spring Harbor Laboratory, NY, 1982.

Kriegler and Botchan, “Enhanced transformation by a simian virus 40 recombinant virus containing a Harvey murine sarcoma virus long terminal repeat,” Mol. Cell. Biol. 3:325, 1983.

Kriegler et al., “Promoter substitution and enhancer augmentation increases the penetrance of the sv40 a gene to levels comparable to that of the harvey murine sarcoma virus ras gene in morphologic transformation,” In: Gene Expression, Alan Liss (Ed.), Hamer and Rosenberg, New York, 1983.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. (eds), Cold Spring Harbor, Cold Spring Harbor Laboratory, 1984.

Kriegler, Perez, Defay, Albert and Liu, “A Novel Form of TNF/Cachectin Is a Cell-Surface Cytotoxix Transmembrane Protein: Ramifications for the Complex Physiology of TNF,” Cell, 53:45, 1988.

Kriegler, Perez, Hardy and Botchan, “Transformation mediated by the sv40 t antigens: separation of the overlapping sv40 early genes with a retroviral vector,” Cell, 38:483, 1984.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kunz, Zimmerman, Heisig, and Heinrich, “Identification of the Promoter Sequences Involved in the Interleukin-6-Dependent Expression of the Rat Alpha-2-Macroglobulin Gene,” Nucl. Acids Res., 17:1121, 1989.

Laird D W, Jordan K, Thomas T, Qin H, Fistouris P, Shao Q. Comparative analysis and application of fluorescent protein-tagged connexins. Microsc Res Tech. Feb. 1, 2001;52(3):263-72.

Lareyre, Thomas, Zheng, Kasper, Ong, Orgebin-Crist, Matusik, “A 5-kilobase pair promoter fragment of the murine epididymal retinoic acid-binding protein gene drives the tissue-specific, cell-specific, and androgen-regulated expression of a foreign gene in the epididymis of transgenic mice,” J. Biol Chem, 274(12):8282-8290, 1999.

Larsen, Harney, and Moore, “Repression medaites cell-type-specific expression of the rat growth hormone gene,” Proc Natl. Acad. Sci. USA., 83:8283, 1986.

Laspia, Rice, and Mathews, “HIV-1 Tat protein increases transcriptional initiation and stabilizes elongation,” Cell, 59:283, 1989.

Latimer, Berger, and Baumann, “Highly conserved upstream regions of the alpha.sub.1-antitryPSIn gene in two mouse species govern liver-specific expression by different mechanisms,” Mol. Cell. Biol., 10:760, 1990.

Laughlin, Cardellichio, and Coon, “Latent infection of kb cells with adeno-associated virus type 2,” J. Virol, 60:515-524, 1986.

Lebkowski, McNally, Okarma, and Lerch, “Adeno-associated virus: a vector system for efficient introduction and integration of DNA into a variety of mammalian cell types,” Mol. Cell. BioL., 8:3988-3996, 1988.

Lee, Mulligan, Berg, and Ringold, “Glucocorticoids Regulate Expression of Dihydrofolate Reductase cDNA in Mouse Mammary Tumor Virus Chimaeric Plasmids,” Nature, 294:228, 1981.

Lee, Wang, Yajima, Jose, Mouradian, “Tissue-specific promoter usage in the D1A dopamine receptor gene in brain and kidney,” DNA Cell Biol, 16(11):1267-75, 1997.

Lehmann, Chiesa, Harris, “Evidence for a six-transmembrane domain structure of presenilin 1,” J. Biol. Chem., 272:12047-12051, 1997.

Levenson et al., “Internal ribosomal entry site-containing retroviral vectors with green fluorescent protein and drug resistance markers,” Human Gene Therapy, 9:1233-1236, 1998.

Levesque, Yu, Nishimura, Zhang, Levesque, Yu, Xu, Liang, Rogaeva, Ikeda, Duthie, Murgolo, Wang, VanderVere, Bayne, Strader, Rommens, Fraser, St. George-Hyslop, “Presenilins interact with armadillo proteins including neural-specific plakophilin-related protein and beta-catenin,” J. Neurochem., 72:999-1008, 1999.

Levinson, Khoury, VanDeWoude, and Gruss, “Activation of SV40 Genome by 72-Base-Pair Tandem Repeats of Moloney Sarcoma Virus,” Nature, 295:79, 1982.

Levitan and Greenwald, “Effects of SEL-12 presenilin on LIN-12 localization and function in caenorhabditis elegans,” Development, 125:3599-3606, 1998.

Li and Greenwald, “Additional evidence for an eight-transmembrane-domain topology for caenorhabditis elegans and human presenilins,” Proc. Natl. Acad. Sci. USA, 95:7109-7114, 1998.

Li and Greenwald, “Membrane topology of the C. elegans SEL-12 presenilin,” Neuron, 17:1015-1021, 1996.

Lin, Cross, Halden, Dragos, Toledano, and Leonard, “Delineation of an enhancerlike positive regulatory element in the interleukin-2 receptor .alpha.-chain gene,” Mol.

Cell. Biol., 10:850, 1990.

Loetscher, Deuschle, Brockhaus, Reinhardt, Nelboeck, Mous, Grunberg, Haass, Jacobsen, “Presenilins are processed by caspase-type proteases,” J. Biol. Chem., 272:20655-20659, 1997.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Natl. Acad. Sci. U.S.A., 83:3609, 1986.

Lusky, Berg, Weiher, and Botchan, “Bovine Papilloma Virus Contains an Activator of Gene Expression at the Distal End of the Early Transcription Unit,” Mol. Cell.

Biol. 3:1108, 1983.

Macejak and Sarnow, “Internal initiation of translation mediated by the 5′ leader of a cellular mRNA,” Nature, 353:90-94, 1991.

Majors and Varmus, “A small region of the mouse mammary tumor virus long terminal repeat confers glucocorticoid hormone regulation on a linked heterologous gene,” Proc. Natl. Acad. Sci. USA, 80:5866, 1983.

Majoul I, Straub M, Hell S W, Duden R, Soling H D. KDEL-cargo regulates interactions between proteins involved in COPI vesicle traffic: measurements in living cells using FRET. Dev Cell. 2001 July; 1(1):139-53.

Maniatis, et al., Molecular Cloning, A Laboratory Manual, Cold Spring Harbor Press, Cold Spring Harbor, N.Y., 1989.

Mann, Mulligan, Baltimore, “Construction of a retrovirus packaging mutant and its use to produce helper-free defective retrovirus,” Cell, 33:153-159, 1983.

McLaughlin, Collis, Hermonat, and Muzyczka, “Adeno-Associated Virus General Transduction Vectors: Analysis of Proviral Structures,” J. Virol., 62:1963-1973, 1988.

McNeall, Sanchez, Gray, Chesterman, and Sleigh, “Hyperinducible Gene Expression From a Metallotionein Promoter Containing Additional Metal-Responsive Elements,” Gene, 76:81, 1989.

Mercken, Takahashi, Honda, Sato, Murayama, Nakazato, Noguchi, Imahori, Takashima, “Characterization of human presenilin 1 using N-terminal specific monoclonal antibodies: Evidence that Alzheimer mutations affect proteolytic processing,” FEBS Lett., 389:297-303, 1996.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Miller, Cai, Krause, “The active site of Serratia endonuclease contains a conserved magnesium-water cluster,” J. Mol. Biol., 288:975-987, 1999.

Miller, Curr. Top. Microbiol. Immunol., 158:1, 1992.

Mordacq and Linzer, “Co-localization of Elements Required for Phorbol Ester Stimulation and Glucocorticoid Repression of Proliferin Gene Expression,” Genes and Dev., 3:760, 1989.

Moreau, Hen, Wasylyk, Everett, Gaub, and Chambon, “The SV40 base-repair repeat has a striking effect on gene expression both in sv40 and other chimeric recombinants,” Nucl. Acids Res., 9:6047, 1981.

Muller and Zahn, “Bleomycin, an antibiotic that removes thymine from double-stranded DNA,” Prog Nucleic Acid Res Mol Biol, 20:21-57, 1977.

Murayama, Tanaka, Palacino, Murayama, Honda, Sun, Yasutake, Nihonmatsu, Wolozin, Takashima, “Direct association of presenilin-1 with beta-catenin,” FEBS Lett., 433, 73-77, 1998.

Musesing, Smith, and Capon, “Regulation of mRNA Accumulation by a Human Immunodeficiency Virus Trans-Activator Protein,” Cell, 48:691, 1987.

Muzyczka, “Use of Adeno-Associated Virus as a General Transduction Vector for Mammalian Cells,” Curr. Top. MicrobioL Immunol., 158:97-129, 1992.

Nabel et al., “Recombinant fibroblast growth factor-1 promotes intimal hyperplasia and angiogenesis in arteries in vivo,” Nature, 362:844-846, 1993.

Naldini, Blomer, Gallay, Ory, Mulligan, Gage, Verma, Trono, “In vivo gene delivery and stable transduction of nondividing cells by a lentiviral vector,” Science, 272(5259):263-267, 1996.

Naruse, Thinakaran, Luo, Kusiak, Tomiata, Iwatsubo, Qian, Ginty, Price, Borchelt, Wong, “Effects of PS1 deficiency on membrane protein trafficking in neurons,” Neuron, 21:1213-1221, 1998.

Ng, Gunning, Liu, Leavitt, and Kedes, “Regulation of the Human Beta-Actin Promoter by Upstream and Intron Domains,” Nuc. Acids Res., 17:601, 1989.

Nicolas and Rubinstein, “Retroviral vectors,” In: Vectors: A survey of molecular cloning vectors and their uses, Rodriguez and Denhardt (eds.), Stoneham: Butterworth, 494-513, 1988.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987.

Nishimura, Yu, Levesque, Zhang, Ruel, Chen, Milman, Holmes, Liang, Kawarai et al., “Presenilin mutations associated with Alzheimer disease cause defective intracellular trafficking of beta-catenin, a component of the presenilin protein complex,” Nature Med., 5:164-169, 1999.

Nomoto, Tatematsu, Takahashi, Osada, “Cloning and characterization of the alternative promoter regions of the human LIMK2 gene responsible for alternative transcripts with tissue-specific expression,” Gene, 236(2):259-71, 1999.

Ondek, Sheppard, and Herr, “Discrete Elements Within the SV40 Enhancer Region Display Different Cell-Specific Enhancer Activities,” EMBO J., 6:1017, 1987.

Ornitz, Hammer, Davison, Brinster, and Palmiter, “Promoter and enhancer elements from the rat elastase i gene function independently of each other and of heterologous enhancers,” Mol. Cell. Biol. 7:3466, 1987.

Palmiter, Chen, and Brinster, “Differential regulation of metallothionein-thymidine kinase fusion genes in transgenic mice and their offspring,” Cell, 29:701, 1982.

Paskind et al., “Dependence of moloney murine leukemia virus production on cell growth,” Virology, 67:242-248, 1975.

PCT Application No. WO 94/09699

PCT Application No. WO 95/06128

PCT Application No. WO 95/07463

Pech, Rao, Robbins, and Aaronson, “Functional identification of regulatory elements within the promoter region of platelet-derived growth factor 2,” Mol. Cell. Biol., 9:396, 1989.

Pelletier and Sonenberg, “Internal initiation of translation of eukaryotic mRNA directed by a sequence derived from poliovirus RNA,” Nature, 334:320-325, 1988.

Perales et al., “Gene transfer in vivo: sustained expression and regulation of genes introduced into the liver by receptor-targeted uptake,” Proc. Natl. Acad. Sci. USA, 91:4086-4090,1994.

Perez-Stable and Constantini, “Roles of fetal γ-globin promoter elements and the adult β-globin 3′ enhancer in the stage-specific expression of globin genes,” Mol. Cell. Biol., 10:1116, 1990.

Picard and Schaffner, “A Lymphocyte-Specific Enhancer in the Mouse Immunoglobulin Kappa Gene,” Nature, 307:83, 1984.

Pinkert, Omitz, Brinster, and Palmiter, “An albumin enhancer located 10 kb upstream functions along with its promoter to direct efficient, liver-specific expression in transgenic mice,” Genes and Dev., 1:268, 1987.

Podlisny, Citron, Amarante, Sherrington, Xia, Zhang, Diehl, Levesque, Fraser, Haass, Koo, Seubert, St. George-Hyslop, Teplow, Selkoe, “Presenilin proteins undergo heterogeneous endoproteolysis between Thr291 and Ala299 and occur as stable N- and C-terminal fragments in normal and Alzheimer brain tissue,” Neurobiol. Dis., 3:325-337, 1997.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Natl. Acad. Sci. U.S.A., 82:1020, 1985.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10:1076, 1990.

Potrykus et al., Mol. Gen. Genet., 199:183-188, 1985.

Potter et al., “Enhancer-dependent expression of human k immunoglobulin genes introduced into mouse pre-B lymphocytes by electroporation,” Proc Nat'l Acad. Sci. USA, 81:7161-7165, 1984.

Prasher, “Using GFP to see the light,” Trends Genet., 11(8):320-3, 1995.

Qian, Jiang, Guan, Singh, Trumbauer, Yu, Chen, Van de Ploeg, Zheng, “Mutant human presenilin 1 protects presenilin 1 null mouse against embryonic lethality and elevates Abetal-42/43 expression,” Neuron, 20, 611-617, 1998.

Queen and Baltimore, “Immunoglobulin Gene Transcription is Activated by Downstream Sequence Elements,” Cell, 35:741, 1983.

Quinn, Farina, Gardner, Krutzsch, and Levens, “Multiple components are required for sequence recognition of the apI site in the gibbon ape leukemia virus enhancer,” Mol. Cell. Biol., 9:4713, 1989.

Ratovitski, Slunt, Thinakaran, Price, Sisodia, Borchelt, “Endoproteolytic processing and stabilization of wild-type and mutant presenilin,” J. Biol. Chem., 272:24536-24541, 1997.

Redondo, Hata, Brocklehurst, and Krangel, “A T-Cell-Specific Transcriptional Enhancer Within the Human T-Cell Receptor .delta. Locus,” Science, 247:1225, 1990.

Reisman and Rotter, “Induced Expression From the Moloney Murine Leukemia Virus Long Terminal Repeat During Differentiation of Human Myeloid Cells is Mediated Through its Transcriptional Enhancer,” Mol. Cell. Biol., 9:3571, 1989.

Resendez Jr., Wooden, and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.

Ridgeway, “Mammalian expression vectors,” In: Vectors: A survey of molecular cloning vectors and their uses. Rodriguez and Denhardt, eds. Stoneham: Butterworth, pp. 467-492, 1988.

Ripe, Lorenzen, Brenner, and Breindl, “Regulatory elements in the 5′ flanking region and the first intron contribute to transcriptional control of the mouse alpha-1-type collagen gene,” Mol. Cell. Biol., 9:2224, 1989.

Rippe, Brenner and Leffert, “DNA-mediated gene transfer into adult rat hepatocytes in primary culture,” Mol. Cell Biol., 10:689-695, 1990.

Rittling, Coutinho, Amarm, and Kolbe, “AP-1/jun-binding Sites Mediate Serum Inducibility of the Human Vimentin Promoter,” Nuc. Acids Res., 17:1619, 1989.

Rosen, Sodroski, and Haseltine, “The location of cis-acting regulatory sequences in the human t-cell lymphotropic virus type III (HTLV-111/LAV) long terminal repeat,” Cell, 41:813, 1988.

Roux et al., “A versatile and potentially general approach to the targeting of specific cell types by retroviruses: Application to the infection of human cells by means of major histocompatibility complex class I and class II antigens by mouse ecotropic murine leukemia virus-derived viruses,” Proc. Natl. Acad. Sci. USA, 86:9079-9083, 1989.

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144, 1988.

Sambrook, Fritsch, Maniatis, Molecular cloning: a laboratory manual. Second Edition, Cold Sprin Harbor: Cold Sprin Harbor Laboratory Press, 1989.

Satake, Furukawa, and Ito, “Biological activities of oligonucleotides spanning the f9 point mutation within the enhancer region of polyoma virus DNA,” J. Virology, 62:970, 1988.

Schaffner, Schirm, Muller-Baden, Wever, and Schaffner, “Redundancy of Information in Enhancers as a Principle of Mammalian Transcription Control,” J. Mol. Biol., 201:81, 1988.

Scheuner, Eckman, Jensen, Song, Citron, Suzuki, Bird, T.D., Hardy, J., Hutton, M., Kukull, W., Larson, E., Levy-Lahad, E., Viitanen, M., Peskind, E., Poorkaj, P., Schellenberg, G., Tanzi, R., Wasco, W., Lannfelt, L., Selkoe, D., and Younkin, S. (1996) Secreted amyloid beta-protein similar to that in the senile plaques of Alzheimer's disease is increased in vivo by the presenilin 1 and 2 and APP mutations linked to familial Alzheimer's disease. Nature Med., 2, 864-870.

Searle, Stuart, and Palmiter, “Building a metal-responsive promoter with synthetic regulatory elements,” Mol. Cell. Biol., 5:1480, 1985.

Seeger, Nordstedt, Petanceska, Kovacs, Gouras, Hahne, Fraser, Levesque, Czemik, George-Hyslop, Sisodia, Thinakaran, Tanzi, Greengard, Gandy, “Evidence for phosphorylation and oligomeric assembly of presenilin 1,” Proc. Natl. Acad. Sci. USA, 94:5090-5094, 1997.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J., 6:1913, 1987.

Sherman, Basta, Moore, Brown, and Ting, “Class II Box Consensus Sequences in the HLA-DR.alpha. Gene: Transcriptional Function and Interaction with Nuclear Proteins,” Mol. Cell. Biol.,9:50, 1989.

Shearnan M S, Beher D, Clarke E E, Lewis H D, Harrison T, Hunt P, Nadin A, Smith A L, Stevenson G, Castro J L. L-685,458, an aspartyl protease transition state mimic, is a potent inhibitor of amyloid beta-protein precursor gamma-secretase activity. Biochemistry. August 1;39(30):8698-704, 2000.

Sisodia, Kim, Thinakaran, “Function and Dysfunction of the Presenilins,” Am J Hum Genet, 65:7-12, 1999.

Sleigh and Lockett, “SV40 Enhancer Activation During Retinoic-Acid-Induced Differentiation of F9 Embryonal Carcinoma Cells,” J. EMBO, 4:3831, 1985.

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J, 2:1193, 1983.

Stabler, Ostrowski, Janicki, Monteiro, “A myristoylated calcium-binding protein that preferentially interacts with the Alzheimer's disease presenilin 2 protein,” J. Cell Biol., 145, 1277-1292, 1999.

Stahl, Diehlmann, T C, “Direct interaction of Alzheimer's disease-related presenilin 1 with armadillo protein p0071,” J. Biol. Chem., 274:9141-9148, 1999.

Steiner, Capell, Pesold, Citron, Kloetzel, Selkoe, Romig, Mendla, Haass, “Expression of Alzheimer's Disease-associated Presenilin-1 Is Controlled by Proteolytic Degradation and Complex Formation,” J. Biol. Chem., 273:32322-32331, 1998.

Steiner, Duff, Capell, Romig, Grim, Lincoln, Hardy, Yu, Picciano, Fechteler, Citron, Kopan, Pesold, Keck, Baader, Tomita, Iwatsubo, Baumeister, Haass, “A loss of function mutation of presenilin-2 interferes with amyloid beta-peptide production and notch signaling,” J. Biol. Chem., 274:28669-28673, 1999.

Steiner, Romig, Pesold, Philipp, Baader, Citron, Loetscher, Jacobsen, Haass, “Amyloidogenic Function of the Alzheimer's Disease-Associated Presenilin 1 in the Absence of Endoproteolysis,” Biochemistry, 38:14600-14605, 1999.

Stephens and Hentschel, “The Bovine Papilloma Virus Genome and its Uses as a Eukaryotic Vector,” Biochem. J., 248:1, 1987.

Struhl and Greenwald, “Presenilin is required for activity and nuclear access of Notch in Drosophila,” Nature, 398:522-525, 1999.

Stuart, Searle, and Palmiter, “Identification of Multiple Metal Regulatory Elements in Mouse Metallothionein-1 Promoter by Assaying Synthetic Sequences,” Nature, 317:828, 1985.

Sullivan and Peterlin, “Transcriptional Enhancers in the HLA-DQ Subregion,” Mol. Cell. Biol., 7:3315, 1987.

Suzuki, Cheung, Cai, Odaka, Otvos Jr., Eckman, Golde, Younkin, “An increased percentage of long amyloid beta protein secreted by familial amyloid beta protein precursor (beta APP717) mutants,” Science, 264:1336-1340, 1994.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Takebe, Seiki, Fujisawa, Hoy, Yokota, Arai, Yoshida, and Arai, “SR.alpha. Promoter: An Efficient and Versatile Mammalian cDNA Expression System Composed of the Simian Virus 40 Early Promoter and the R-U5 Segment of Human T-Cell Leukemia Virus Type 1 Long Terminal Repeat,” Mol. Cell. Biol., 8:466, 1988.

Tanahashi and Tabira, “Isolation of human delta-catenin and its binding specificity with presenilin 1,” Neuroreport, 10:563-568, 1999.

Tavernier, Gheysen, Duerinck, Can Der Heyden, and Fiers, “Deletion Mapping of the Inducible Promoter of Human IFN-beta Gene,” Nature, 301:634, 1983. Taylor and Kingston, “E1A Trans-Activation of Human HSP70 Gene Promoter Substitution Mutants is Independent of the Composition of Upstream and TATA Elements,” Mol. Cell. Biol., 10:176, 1990.

Taylor and Kingston, “Factor Substitution in a Human HSP70 Gene Promoter: TATA-Dependent and TATA-Independent Interactions,” Mol. Cell. Biol., 10: 165, 1990.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Tesco, Kim, Diehlmann, Beyreuther, Tanzi, “Abrogation of the presenilin 1/beta-catenin interaction and preservation of the heterodimeric presenilin 1 complex following caspase activation,” J. Biol. Chem., 273:33909-33914, 1998.

Thiesen, Bosze, Henry, and Charnay, “A DNA Element Responsible for the Different Tissue Specificities of Friend and Moloney Retroviral Enhancers,” J. Virology, 62:614, 1988.

Thinakaran, “The role of presenilins in Alzheimer's disease,” J. Clin Invest, 104:1321-1327, 1999.

Thinakaran, Borchelt, Lee, Slunt, Spitzer, Kim, Ratovitsky, Davenport, Nordstedt, Seeger, Hardy, Levey, Gandy, Jenkins, Copeland, Price, Sisodia, “Endoproteolysis of presenilin 1 and accumulation of processed derivatives in vivo,” Neuron, 17:181-190, 1996.

Thinakaran, Harris, Ratovitski, Davenport, Slunt, Price, Borchelt, Sisodia, “Evidence that levels of presenilins (PS1 and PS2) are coordinately regulated by competition for limiting cellular factors,” J. Biol. Chem., 272:28415-28422, 1997.

Thinakaran, Regard, Bouton, Harris, Price, Borchelt, Sisodia, “Stable association of presenilin derivatives and absence of presenilin interactions with APP,” Neurobiol. Dis., 4:438-453, 1998.

Thinakaran, Teplow, Siman, Greenberg, Sisodia, “Metabolism of the “Swedish” amyloid precursor protein variant in neuro2a (N2a) cells. Evidence that cleavage at the “beta-secretase” site occurs in the golgi apparatus,” J. Biol. Chem., 271:9390-9397, 1996.

Tomita, Maruyama, Saido, Kume, Shinozaki, Tokuhiro, Capell, Walter, Grunberg, Haass, Iwatsubo, Obata, “The presenilin 2 mutation (N141I) linked to familial Alzheimer disease (Volga German families) increases the secretion of amyloid beta protein ending at the 42nd (or 43rd) residue,” Proc. Natl. Acad. Sci. USA, 94:2025-2030, 1997.

Tomita, Takikawa, Koyama, Morohashi, Takasugi, Saido, T. C., Maruyama, K., and Iwatsubo, T. (1999) C terminus of presenilin is required for overproduction of amyloidogenic Ab42 through stabilization and endoproteolysis of presenilin. J. Neurosci., 19, in press.

Tratschin, West, Sandbank, and Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Treisman, “Transient Accumulation of c-fos RNA Following Serum Stimulation Requires a Conserved 5′ Element and c-fos 3′ Sequences,” Cell, 42:889, 1986.

Tronche, Rollier, Bach, Weiss, and Yaniv, “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759, 1989.

Tronche, Rollier, Herbomel, Bach, Cereghini, Weiss, and Yaniv, “Anatomy of the Rat Albumin Promoter,” Mol. Biol. Med., 7:173, 1990.

Trudel and Constantini, “A 3′ Enhancer Contributes to the Stage-Specific Expression of the human Beta-Globin Gene,” Genes and Dev., 6:954, 1987.

Tsumaki et al., “Modular arrangement of cartilage- and neural tissue-specific cis-elements in the mouse alpha2(XI) collagen promoter,” J Biol Chem. 273(36):22861-22864, 1998.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

U.S. Pat. No. 4,683,202

U.S. Pat. No. 4,797,368

U.S. Pat. No. 5,139,941

U.S. Pat. No. 5,925,565

U.S. Pat. No. 5,928,906

U.S. Pat. No. 5,928,906

U.S. Pat. No. 5,935,819

U.S. Pat. No. 5,994,136

U.S. Pat. No. 6,013,516

U.S. Pat. No. 6,172,188

U.S. Pat. No. 4,683,202

U.S. Pat. No. 4,684,611

U.S. Pat. No. 4,797,368

U.S. Pat. No. 4,879,236

U.S. Pat. No. 4,952,500

U.S. Pat. No. 5,139,941

U.S. Pat. No. 5,302,523

U.S. Pat. No. 5,322,783

U.S. Pat. No. 5,384,253

U.S. Pat. No. 5,464,765

U.S. Pat. No. 5,508,184

U.S. Pat. No. 5,538,877

U.S. Pat. No. 5,538,880

U.S. Pat. No. 5,550,318

U.S. Pat. No. 5,580,859

U.S. Pat. No. 5,589,466

U.S. Pat. No. 5,591,616

U.S. Pat. No. 5,610,042

U.S. Pat. No. 5,656,610

U.S. Pat. No. 5,702,932

U.S. Pat. No. 5,780,448

U.S. Pat. No. 5,789,215

U.S. Pat. No. 5,871,986

U.S. Pat. No. 5,925,565

U.S. Pat. No. 5,935,819

U.S. Pat. No. 5,945,100

U.S. Pat. No. 5,981,274

U.S. Pat. No. 5,994,136

U.S. Pat. No. 5,994,624

U.S. Pat. No. 6,013,516

Vannice and Levinson, “Properties of the Human Hepatitis B Virus Enhancer: Position Effects and Cell-Type Nonspecificity,” J. Virology, 62:1305, 1988.

Vasseur, Kress, Montreau, and Blangy, “Isolation and Characterization of Polyoma Virus Mutants Able to Develop in Multipotential Murine Embryonal Carcinoma Cells,” Proc Natl. Acad. Sci. U.S.A., 77:1068, 1980.

Wagner et al., Science, 260:1510-1513, 1990.

Walter, Capell, Grunberg, Pesold, Schindzielorz, Prior, Podlisny, Fraser, Hyslop, Selkoe, Haass, “The Alzheimer's disease-associated presenilins are differentially phosphorylated proteins located predominantly within the endoplasmic reticulum,” Mol. Med., 2:673-691, 1996.

Wang and Calame, “SV40 enhancer-binding factors are required at the establishment but not the maintenance step of enhancer-dependent transcriptional activation,” Cell, 47:241, 1986.

Watowich and Morimoto, “Complex regulation of heat shock- and glucose-responsive genes in human cells,” Mol. Cell. Biol., 8:393-405, 1988.

Weber, De Villiers, and Schaffner, “An SV40 Enhancer Trap Incorporates Exogenous Enhancers or Generates Enhancers From its Own Sequences,” Cell, 36:983, 1984.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Weiss T S, Chamberlain C E, Takeda T, Lin P, Hahn K M, Farquhar MG.Galpha i3 binding to calnuc on Golgi membranes in living cells monitored by fluorescence resonance energy transfer of green fluorescent protein fusion proteins. Proc Natl Acad Sci U S A. Dec. 18, 2001;98(26):14961-6.

Wilson et al., “Implantation of vascular grafts lined with genetically modified endothelial cells,” Science, 244:1344-1346, 1989.

Wiltfang, Smimov, Schnierstein, Kelemen, Matthies, Klafki, Staufenbiel, Huther, Ruther, Kornhuber, “Improved electrophoretic separation and immunoblotting of beta-amyloid (A beta) peptides 1-40, 1-42, and 1-43,” Electrophoresis, 18:527-532, 1997.

Winoto and Baltimore, “αβ-lineage-specific Expression of the a T-Cell Receptor Gene by Nearby Silencers,” Cell, 59:649, 1989.

Wolfe, Xia, Ostaszewski, Diehl, Kimberly, Selkoe, (1999) Two transmembrane aspartates in presenilin-1 required for presenilin endoproteolysis and gamma-secretase activity. Nature, 398:513-517.

Wong et al., “Appearance of β-lactamase activity in animal cells upon liposome mediated gene transfer,” Gene, 10:87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Wu and Wu, Biochem., 27:887-892, 1988.

Wu, Squire, Song, Weksberg, “Promoter-dependent tissue-specific expressive nature of imprinting gene, insulin-like growth factor II, in human tissues,” Biochem Biophys Res Commun. 233(1):221-226, 1997.

Yang, Burkholder, Roberts, Martinell and McCabe, “In vivo and in vitro gene transfer to mammalian somatic cells by particle bombardment,” Proc Nat'l Acad Sci. USA, 87:9568-9572, 1990.

Ye, Lukinova, Fortini, “Neurogenic phenotypes and altered Notch processing in Drosophila Presenilin mutants,” Nature, 398:525-529, 1999.

Yu, Chen, Levesque, Nishimura, Zhang, Levesque, Rogaeva, Xu, Liang, Duthie, George-Hyslop, Fraser, “The Presenilin 1 Protein Is a Component of a High Molecular Weight Intracellular Complex That Contains beta-Catenin,” J. Biol. Chem., 273:16470-16475, 1998.

Yutzey, Kline, and Konieczny, “An Internal Regulatory Element Controls Troponin I Gene Expression,” Mol. Cell. Biol., 9:1397, 1989.

Zhang, Han, McKeel, Goate, Wu, “Interaction of presenilins with the filamin family of actin-binding proteins,” J. Neurosci., 18:914-922, 1998.

Zhang, Hartmann, Do, Abramowski, Sturchler-Pierrat, Staufenbiel, Sommer, van de Wetering, Clevers, Saftig, De Strooper, He, Yankner, “Destabilization of beta-catenin by mutations in presenilin-1 potentiates neuronal apoptosis,” Nature, 395:698-702, 1998.

Zhang, Kang, Xia, Okochi, Mori, Selkoe, Koo, “Subcellular distribution and turnover of presenilins in transfected cells,” J. Biol. Chem., 273:12436-12442, 1998.

Zhao-Emonet, Boyer, Cohen, Klatzmann, “Deletional and mutational analyses of the human CD4 gene promoter: characterization of a minimal tissue-specific promoter,” Biochim Biophys Acta, 1442(2-3): 109-119, 1998.

Zhou, Liyanage, Medina, Ho, Simmons, Lovett, Kosik, “Presenilin 1 interaction in the brain with a novel member of the Armadillo family,” Neuroreport, 8:2085-2090, 1997.

Zufferey, Nagy, Mandel, Naldini, Trono, “Multiply attenuated lentiviral vector achieves efficient gene delivery in vivo,” Nat Biotechnol, 15(9):871-875, 1997.

What is claimed is:

1. A method for identifying candidate substances that change the levels of accumulation of a protein comprising:
a) obtaining a cell expressing a chimeric polypeptide comprising a polypeptide region of the protein linked to at least one marker gene product region;
b) exposing the cell to a candidate substance; and
c) determining any change in a level of the chimeric protein subsequent to exposing the cell with the candidate substance.

2. The method of claim 1, further comprising assaying the level of the chimeric protein using the marker gene product.

3. The method of claim 1, wherein the protein is an unstable protein.

4. The method of claim 3, wherein the unstable protein is a presenilin protein, an amyloid precursor protein, or an amyloid precursor protein derivative.

5. The method of claim 3, wherein the unstable protein is a presenilin protein.

6. The method of claim 5, wherein the presenilin protein is PS1.

7. The method of claim 5, wherein the presenilin protein is PS2.

8. The method of claim 1, wherein the protein is further defined as a polytopic membrane protein.

9. The method of claim 9, wherein the polytopic protein further comprises at least one co-factor.

10. The method of claim 9, wherein the polytopic protein is a ligand-gated ion channel or a voltage-gated ion channel.

11. The method of claim 10, wherein the polytopic protein is a ligand-gated ion channel and is further defined as a nicotinic acetylcholine receptor, a GABA receptor, or a glycine receptor.

12. The method of claim 10, wherein the polytopic protein is a voltage-gated ion channel and is further defined as a voltage-gated Na²⁺ channel, a voltage-gated K⁺ channel, or a voltage-gated Ca²⁺ channel.

13. The method of claim 1, wherein the change is an increase in the level of accumulation of said protein.

14. The method of claim 1, wherein the change is a decrease in the level of accumulation of said protein.

15. The method of claim 1, wherein the candidate substance is a chemical compound.

16. The method of claim 1, wherein the candidate substance is a protein.

17. The method of claim 16, further comprising isolation of the protein candidate substance.

18. The method of claim 1, wherein the candidate substance is a pharmacological compound.

19. The method of claim 1, wherein the candidate sub stanc e is a nucleic acid.

20. The method of claim 19, further defined as comprising transfecting the cell with the nucleic acid.

21. The method of claim 19, wherein the nucleic acid is a cDNA.

22. The method of claim 19, wherein the nucleic acid is a genomic DNA.

23. The method of claim 1, further defined as comprising contacting the cell with said candidate substance.

24. The method of claim 1, further defined as comprising injecting the cell with the candidate substance.

25. The method of claim 1, further defined as comprising administering the candidate substance to the cell.

26. The method of claim 1, wherein the marker gene product is an fluorescent gene product.

27. The method of claim 26, wherein the fluorescent gene product is a yellow fluorescent protein (YFP), a green fluorescent protein (GFP), a blue fluorescent protein (BFP), or a red fluorescent protein (RFP).

28. The method of claim 1, wherein the marker gene product is an antibiotic resistance gene product.

29. The method of claim 28, wherein the antibiotic resistance gene product is further defined as one that confers antibiotic resistance by binding stoichiometrically to an antibiotic.

30. The method of claim 29, wherein the antibiotic resistance gene product is selected from the group consisting of a bleomycin resistance gene product, a zeocin resistance gene product, a zorbamycine resistance gene product, a victomycin resistance gene product, a platomycin resistance gene product, a tallysomycin resistance gene product, a SF 1771 resistance gene product, a SF 1961 resistance gene product, and a YA 56 resistance gene product.

31. The method of claim 29, wherein the antibiotic resistance gene product is the bleomycin resistance gene product.

32. The method of claim 29, wherein the antibiotic resistance gene is the zeocin resistance gene.

33. The method of claim 29, wherein the determining comprises an antibiotic selection assay.

34. The method of claim 33, wherein the antibiotic selection assay comprises selection with a higher concentration of the antibiotic.

35. A method for identifying candidate substances that change the levels of accumulation of an unstable protein comprising
a) obtaining a cell expressing a chimeric polypeptide comprising a polypeptide of the unstable protein linked to at least one marker gene product;
b) exposing the cell to a candidate substance; and
c) determining any change in a level of the chimeric protein subsequent to exposing the cell with the candidate substance.

36. The method of claim 35, further comprising assaying the level of the chimeric protein using the marker gene product.

37. The method of claim 35, wherein the protein is a presenilin protein.

38. The method of claim 35, wherein the marker gene product is an antibiotic resistance gene product.

39. The method of claim 38, wherein the antibiotic resistance gene product is a bleomycin resistance gene product.

40. The method of claim 39, wherein the bleomycin resistance gene product is the Ble protein.

41. The method of claim 35, wherein the marker gene product is an green fluorescent gene product, a yellow fluorescent gene product, a blue fluorescent gene product, or a red fluorescent gene product.

42. The method of claim 41, wherein said determining measures the level of the green fluorescent gene product, the yellow fluorescent gene product, the blue fluorescent gene product or the red fluorescent gene product.

43. A method for identifying candidate substances that change the levels of accumulation of a presenilin protein comprising
a) obtaining a cell expressing a chimeric presenilin polypeptide comprising a presenilin polypeptide linked to a bleomycin resistance gene product;
b) exposing the cell to a candidate substance; and
c) determining any change in a level of the chimeric presenilin subsequent to exposing the cell with the candidate substance.

44. The method of claim 43, wherein the chimeric polypeptide further comprises a fluorescent protein.

45. The method of claim 44, wherein the determining comprises measuring fluorescence.

46. The method of claim 43, wherein the determining comprises an antibiotic selection assay.

47. The method of claim 46, wherein the antibiotic is bleomycin.

48. The method of claim 46, wherein the antibiotic selection assay is performed at a higher level of the antibiotic.

49. A method for identifying candidate substances that change the levels of accumulation of a presenilin protein comprising:
a) obtaining a cell expressing a chimeric presenilin polypeptide comprising a presenilin polypeptide linked to a fluorescent protein gene product;
b) exposing the cell to a candidate substance; and
c) determining any change in a level of the chimeric presenilin subsequent to exposing the cell with the candidate substance.

50. The method of claim 49, wherein the fluorescent protein gene product is a green fluorescent gene product.

51. The method of claim 49, wherein the fluorescent protein gene product is a yellow fluorescent gene product.
